(19) United States
(12) Patent Application Publication
(10) Pub. No.: US 2024/0182512 A1
(43) Pub. Date: Jun. 6, 2024

(54) N-(2-AMINOETHYL)MORPHOLINE-BASED RNA ANALOGS, METHOD FOR THE PREPARATION AND USE THEREOF

(71) Applicant: Uniwersytet Warszawski, Warszawa (PL)

(72) Inventors: Adam MAMOT, Warszawa (PL); Pawel SIKORSKI, Warszawa (PL); Joanna KOWALSKA, Warszawa (PL); Jacek JEMIELITY, Warszawa (PL)

(73) Assignee: Uniwersytet Warszawski, Warszawa (PL)

(21) Appl. No.: 18/281,352

(22) PCT Filed: Mar. 10, 2022
(86) PCT No.: PCT/PL2022/050013

§ 371 Date: Sep. 11, 2023

(30) Foreign Application Priority Data
Mar. 10, 2021 (PL) P.437263

(51) Int. Cl. C07H 21/02

(52) U.S. Cl. CPC C07H 21/02

(57) ABSTRACT

The subject of the invention is RNA analogs, their preparation and application, inter alia, in microscopic observations, study of the gene expression process and monitoring of enzyme activity.

### STATE OF THE ART

The methods of selective covalent modifiction of RNA molecules have many applications, including detection, localization, isolation and sequencing of RNA, study of gene expression process, the immune system, cell development, or molecular dynamics. Depending on the desired effect and the adopted experimental strategy, one of the following methods of RNA modification can be used: modification by a reaction with a catalytic nucleic acid (DNAzyme or RNAzyme), modification by an enzymatic reaction, and modification by a chemical reaction. Catalytic nucleic acids, such as DNAzymes and RNAzymes, are (often synthetic) molecules that, thanks to their sequence, are able to catalyze a given chemical reaction. DNAzymes and RNAzymes, such as 10DM24, FH14, or FJ1, modify RNA by creating a 2′-5′-phosphodiester bond resulting from reacting the 5′ triphosphate group of a functionalized nucleotide analog with the 2′-hydroxyl group of adenosine contained in the target RNA sequence and defined by a catalytic nucleic acid sequence [1-4]. Alternatively, it is possible to modify the 3′-hydroxyl group of the terminal nucleotide of the target RNA or DNA with ribozymes with polymerase activity [5]. Enzymes such as ligases, polymerases, or transferases can be used to selectively modify RNA by an enzymatic reaction. The enzyme uses a functionalized analog of the natural substrate or cofactor to carry out the reaction. Using the bacteriophage T4 RNA ligase, a phosphodiester bond can be created between the 3′-OH group of the target RNA and the 5′-phosphate group of a functionalized 5′,3′-nucleotide diphosphate (pNp) analog [6-7]. RNA polymerases, such as bacteriophage T7 RNA polymerase or polyA polymerase, use functionalized analogs of nucleotide triphosphates, allowing modification of the terminal regions (at the 5′ or 3′-ends) or internal nucleotides of the target RNA [8-12]. From the group of transferases, the most commonly used are methyltransferases, such as Ecm1, which use functionalized analogs of the cofactor S-adenosylmethionine (SAM) to modify the positions N2 of guanosine, N7 of guanosine, N6 of adenosine, and 3′-hydroxyl within the target RNA [13].

To modify RNA by chemical reaction, inherently occurring functional groups, such as 2′- and 3′-hydroxyl groups, amino, amide, imino, carbonyl and enol groups on nitrogenous bases, or phosphate groups can be used. Their use to carry out a selective chemical reaction is challenging due to the high molecular weight of RNA, similar reactivity of many of the groups mentioned, and the possibility of the breakdown of phosphodiester and N-glycosidic bonds under drastic conditions. These problems can be eliminated by introducing unnatural functional groups that can participate in rapid and selective chemical reactions (including bioorthogonal groups). Unnatural functional groups may be introduced in the course of chemical nucleic acid synthesis, for example solid phase synthesis using phosphoramidite chemistry. The main limitation of this approach is the length limit of the resulting polynucleotide chain. Synthesis of over one hundred nucleotide-long DNA and RNA molecules is challenging to the extent that chemical synthesis of two hundred nucleotide-long RNA is virtually impossible. Therefore, only enzymatic or chemoenzymatic methods have been used so far to modify larger RNA molecules, for example protein-coding RNA (mRNA) that are 200-10,000 nucleotides long.

A unique method of RNA modification relies on chemical oxidation of the ribose 2′,3′-cis-diol with metaperiodic acid (HIO₄) salt and subsequent amination or reductive amination reaction. As a result, the 3′-terminal ribose in the RNA is converted to a dihydroxymorpholine or morpholine analog, which may have different functional groups, depending on the structure of the amine derivative used in the amination or reductive amination (FIG. 1A). This approach was used to modify the 3′ terminus of RNA [14-21] with hydrazine analogs and aliphatic amines, whereas amine derivatives exhibit much lower reactivity than hydrazine and hydrazide derivatives (FIG. 1B). Therefore, hydrazine and hydrazide derivatives are most often used for RNA 3′ end modification, although their synthesis is more demanding than synthesis of amines, and the resulting conjugates have lower chemical stability.

The object of the present invention is to provide methods and compounds that eliminate the above-described problems related to the chemical modification of RNA molecules. In particular, it is an object of the invention to solve the problem of low reactivity of the amine derivatives used in reductive amination leading to a morpholine analog obtained by oxidation of the 2′,3′-cis-diol of the ribose at the 3′ terminus of the RNA.

### THE ESSENCE OF THE INVENTION

The subject of the invention is the RNA analog of formula 1:

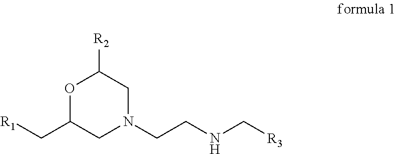

wherein:

### R₁ is:

an RNA chain of formula 2a:

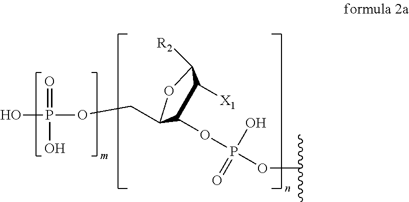

wherein:
n is a natural number in the range from 1 to 10,000,
m is a natural number in the range from 0 to 3,
each X₁ is independently selected among of: OH or OCH₃,
   or an RNA chain of formula 2b:

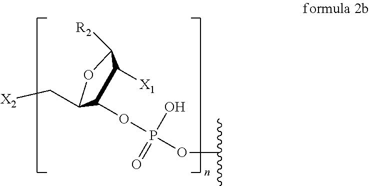

wherein:
n is a natural number in the range from 1 to 10000,
each X₁ is independently selected among of: OH or OCH₃,
X₂ is N₃ or

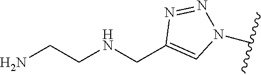 [SMILES: *n1cc(CNCCN)nn1]

group,
or an RNA chain of formula 2c:

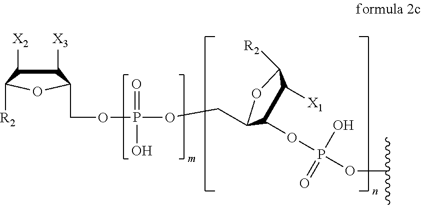

wherein:
n is a natural number in the range from 1 to 10000,
m is a natural number in the range from 1 to 4,
each X₁ is independently selected among of: OH or OCH₃
X₂ and X₃ are independently: OH, OCH₃,

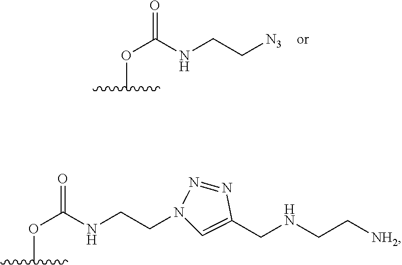 [SMILES: *OC(=O)NCCN=[N+]=[N-] ; *OC(=O)NCCn1cc(CNCCN)nn1]

each R₂ is independently selected among of: a natural or modified purine or pyrimidine nitrogenous base, preferably selected from:

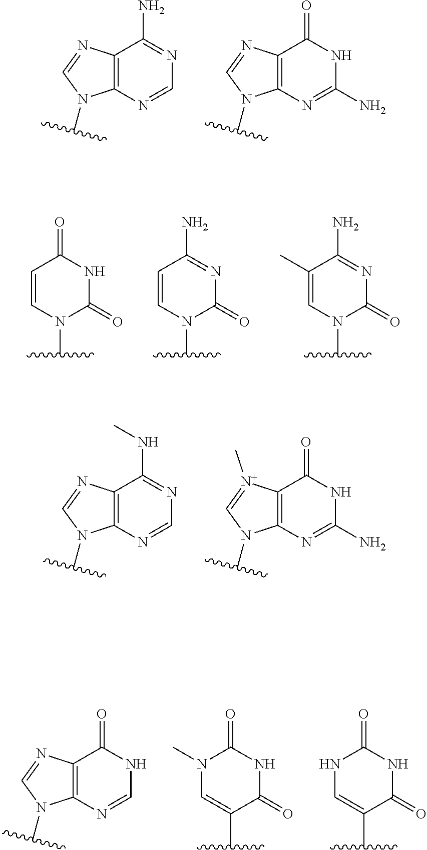

R₃ is a functional group comprising of:
a bioorthogonal group of the formula 3a:

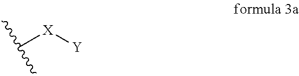

wherein
Y is NH₂, N₃ or

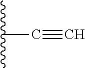 [SMILES: *C#C]

group
or a substituent having the structure of a fluorophore from the cyanine group of formula 3b:

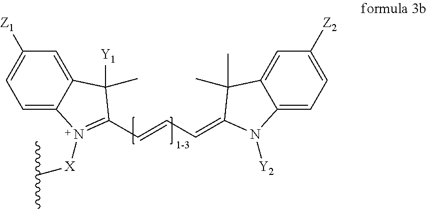

wherein:
Y₁ and Y₂ are independently: CH₃, (CH₂)₃SO₃H or (CH₂)₄SO₃H,
Z₁ and Z₂ are independently: SO₃H or H,
   or a substituent having the structure of a fluorophore from the rhodamine or fluorescein group of formula 3c:

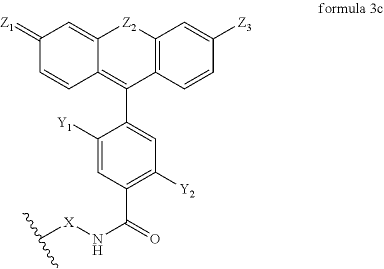

wherein:
Y₁ and Y₂ are independently: SO₃H, OCH₃, OH, COOH or H,
Z₁ and Z₂ are independently: NH or O,
Z₃ is NH₂ or OH group,
   or a substituent having the structure of a fluorophore from the rhodamine group of formula 3d:

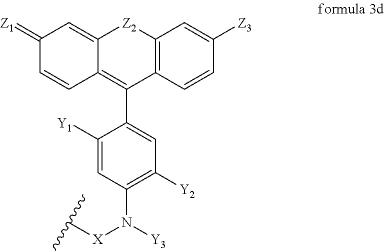

wherein:
Y₁ and Y₂ are independently: SO₃H, OCH₃, OH, COOH or H,
Y₃ is CH₂CH₃, CH₃ or H group,
Z₁ and Z₂ are independently: NH or O,
Z₃ is NH₂ or OH group,
   or a substituent having an affinity tag structure of formula 3e:

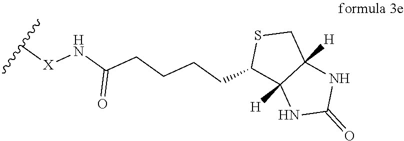

or a substituent having a nucleic acid structure of formula 3f:

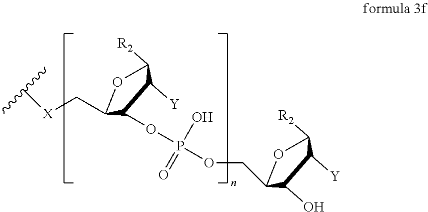

wherein:
each Y is independently selected among of: OCH₃, OH or H,
n is a natural number from 1 to 30,
R₂ is a nitrogenous base as above,
   or a substituent having a nucleic acid structure of formula 3g:

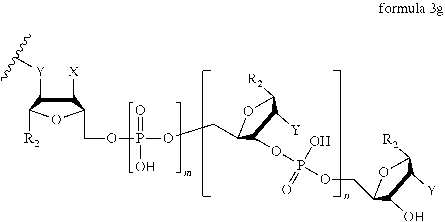

wherein:
each Y is independently selected among of: OCH₃, OH or H group,
m is a natural number in the range from 1 to 4,
n is a natural number in the range from 1 to 30,
R₂ is nitrogenous base as above,
   or a nucleic Arid of the formula 3h.

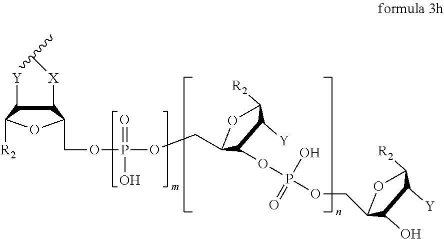

wherein:
each Y is independently selected among of: OCH₃, OH or H group,
m is a natural number from 1 to 4,
n is a natural number from 1 to 30,
R₂ is nitrogenous base as above,
   wherein in the above formulas (3a to 3h) X is a linker of formula being any group or a serial combination of many of the following groups:

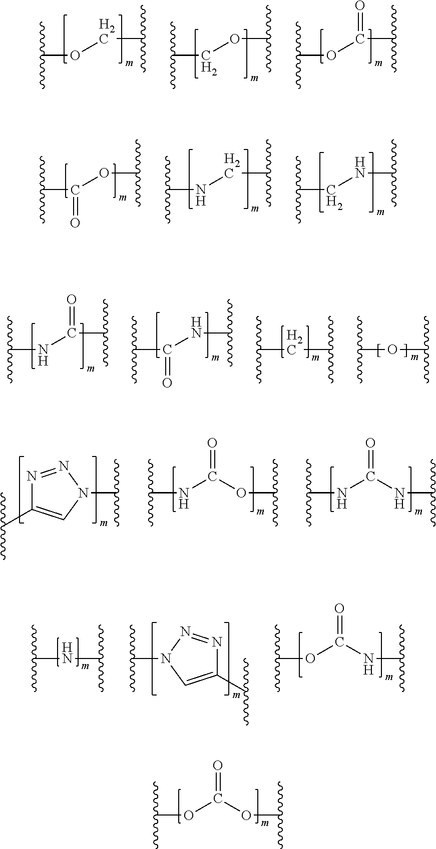

wherein m is a natural number ranging from 1 to 10.

Another object of the invention is a method for the preparation of an RNA analog of formula 1 as defined above, characterized in that the solution of RNA of formula 4:

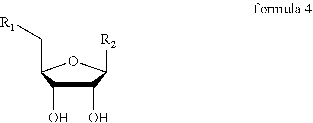

is subjected to successive:
(i) incubation with metaperiodic acid HIO₄, its salt, a solution of metaperiodic acid HIO₄ or its salt, to provide a compound of formula 5:

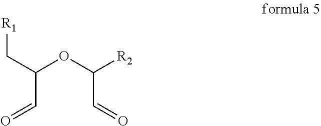

(ii) incubation in a reducing medium with an ethylenediamine analog of formula 6:

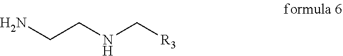

to give an RNA analog of formula 1:

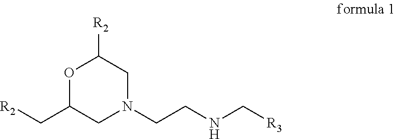

wherein the meaning of the groups R₁, R₂ and R₃ in the above formulas is defined in claim 1.

Preferably, steps (i) and (ii) are carried out in one reactor.

Preferably, the RNA is:

a compound of formula 4a:

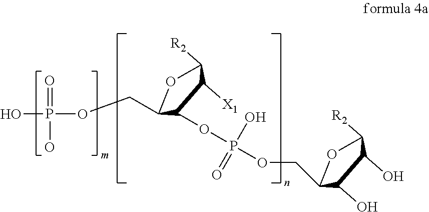

wherein:
n is a natural number in the range from 1 to 10,000,
m is a natural number in the range from 0 to 3,
each X₁ is independently selected among of: OCH₃ or OH,
   or a compound of formula 4b:

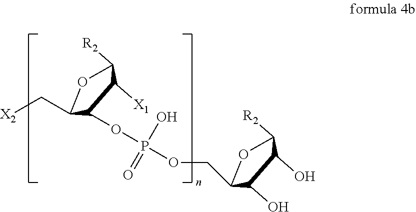

wherein:
n is a natural number in the range from 1 to 10,000,
each X₁ is independently selected among of: OH or CH₃ group,
X₂ is N₃ or

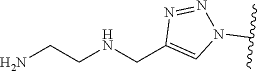 [SMILES: *n1cc(CNCCN)nn1]

group,
or a compound of formula 4c:

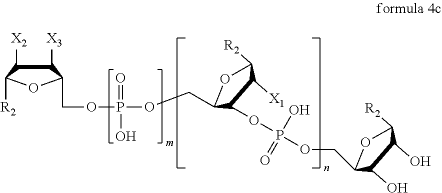

wherein:
n is a natural number in the range from 1 to 10000,
m is a natural number in the range from 1 to 4,
each X₁ is independently selected among of: OH or OCH₃
X₂ and X₃ are independently: OH, OCH₃,

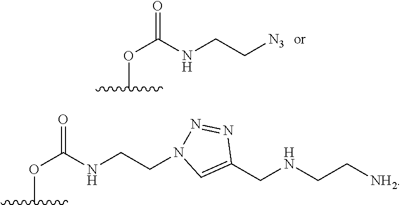 [SMILES: *OC(=O)NCCN=[N+]=[N-] ; *OC(=O)NCCn1cc(CNCCN)nn1]

Preferably, step (i) is carried out in the presence of NaIO₄, preferably at a concentration of 1.0 to 1.5 mM, at a temperature below 40° C., and at the RNA concentration of 1 to 100 μM.

Preferably, step (ii) is carried out in the presence of a KH₂PO₄ buffer, preferably at pH 5.5-7.5, NaBH₃CN reducing agent at a concentration not exceeding 100 mM, preferably at a concentration of 20 mM, and an ethylenediamine analog at a concentration of 1-10 mM.

Preferably, the obtained RNA analog is isolated from the reaction mixture by a known method of RNA isolation, preferably by precipitation of the RNA salt in alcohol or by HPLC.

Another object of the invention is an ethylenediamine analog of formula 7:

wherein R is:

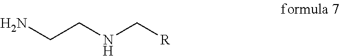

a substituent having the structure of a fluorophore from the cyanine group of formula 7a:

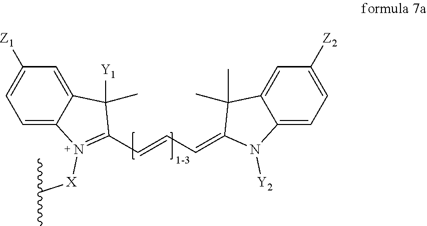

wherein:
Y₁ and Y₂ are independently: CH₃, (CH₂)₃SO₃H or (CH₂)₄SO₃H,
Z₁ and Z₂ are independently: SO₃H or H,
   or a substituent having the structure of a fluorophore from the rhodamine or fluorescein group of formula 7b:

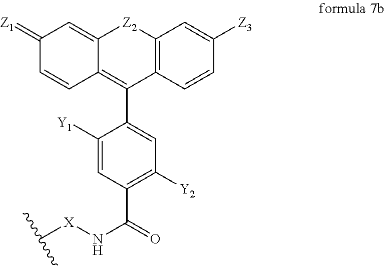

wherein:
Y₁ and Y₂ are independently: SO₃H, OCH₃, OH, COOH or H,
Z₁ and Z₂ are independently: NH or O,
Z₃ is NH₂ or OH group,
   or a substituent having the structure of a fluorophore from the rhodamine group of formula 7c:

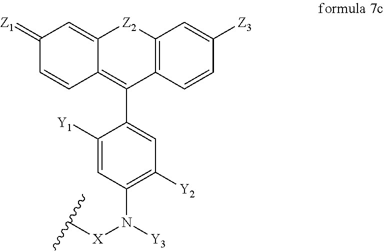

wherein:
Y₁ and Y₂ are independently: SO₃H, OCH₃, OH, COOH or H,
Y₃ is CH₂CH₃, CH₃ or H group,
Z₁ and Z₂ are independently: NH or O,
Z₃ is NH₂ or OH group,
   or a substituent having an affinity tag structure of formula 7d:

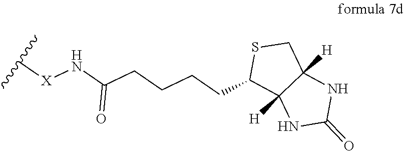

or a substituent having a nucleic acid structure of formula 7e:

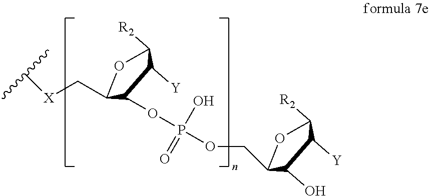

wherein:
Y is independently OCH₃, OH or H group,
n is a natural number in the range from 1 to 30,
R₂ is nitrogenous base as above,
   or a substituent having a nucleic acid structure of formula 7f:

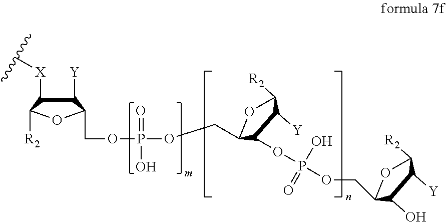

wherein:
each Y is independently selected among of: OCH₃, OH or H group,
m is a natural number in the range from 1 to 4,
n is a natural number in the range from 1 to 30,
R₂ is nitrogenous base as above,
   or a substituent having a nucleic acid structure of formula 7g:

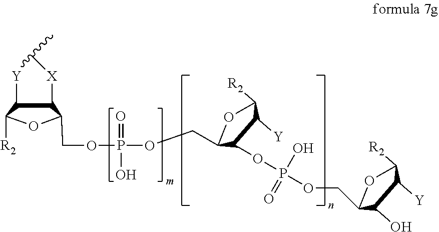

wherein:
each Y is independently selected among of: OCH₃, OH or H group,
m is a natural number in the range from 1 to 4,
n is a natural number in the range from 1 to 30,
R₂ is nitrogenous base as above,
   wherein in the above formulas (7a to 7g) X is a linker of formula being any group or a serial combination of many of the following groups:

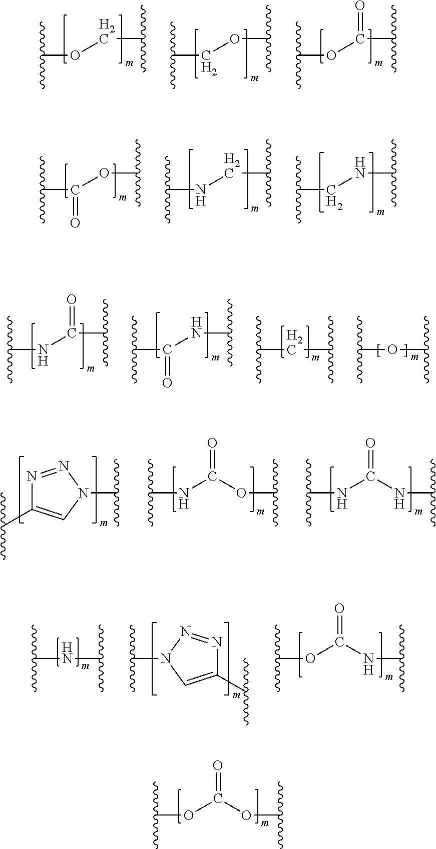

wherein m is a natural number ranging from 1 to 10.

Preferably, the ethylenediamine analog according to the invention is selected from compounds of the formulas:

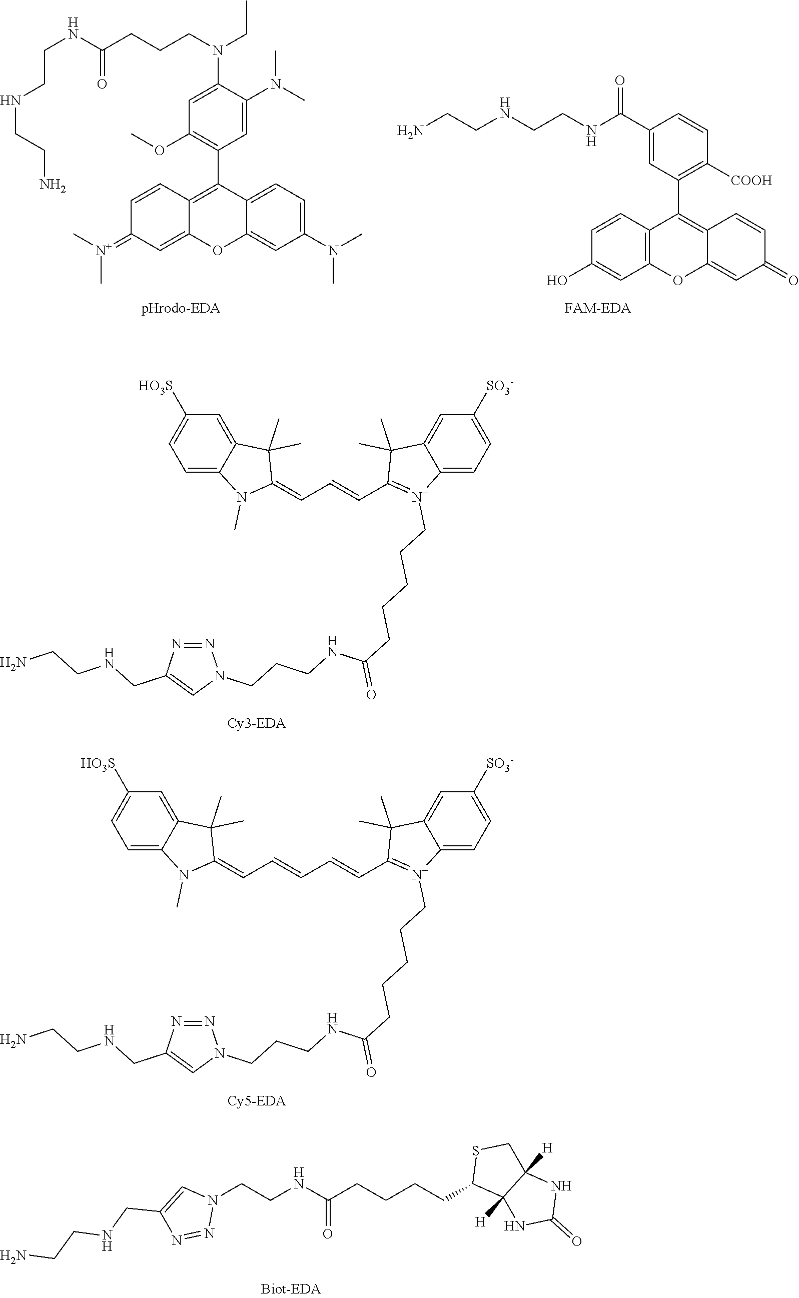

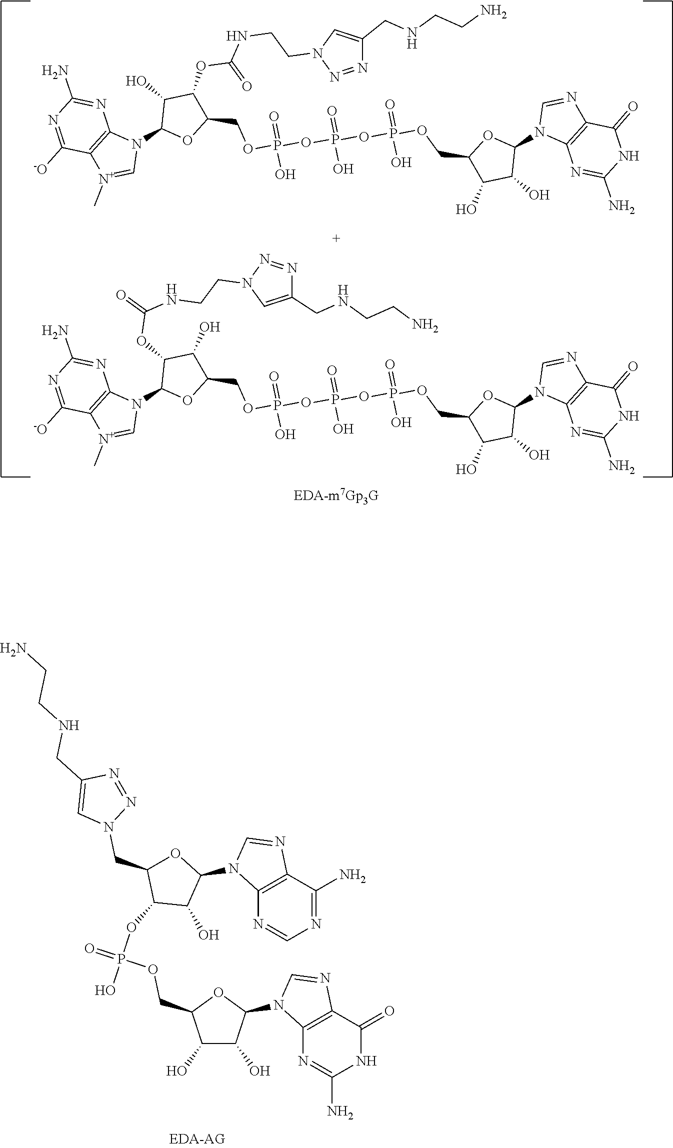

The disclosed N-(2-aminoethyl)morpholine-based RNA analogs are analogs of nucleic acid molecules (linear nucleotide polymers, polynucleotides) in which one of the nucleotides (monomers) is replaced with a unique N-(2-aminoethyl)morpholine moiety (according to formula 1). The moiety has three main substituents (R₁, R₂, and R₃ according to formula 1): the RNA chain (R₁), the nitrogenous base (R₂) and the functional substituent (R₃). The RNA chain (R₁ according to formula 1) is a biopolymer in which monomers (ribonucleotides) are made of pentose (sugar residue) linked to a nitrogenous base and linked to sugar residues of neighboring monomers by phosphodiester bonds. The substituent (R₁) may be derived from chemical synthesis (for example solid phase synthesis using phosphoramidite chemistry) as well as enzymatic synthesis (for example a transcription reaction catalyzed by RNA polymerase), therefore the substituent (R₁) includes RNA chains with a wide range of degree of polymerization (from 1 up to 10,000 nucleotides), or RNA chains containing modifications in the area of a sugar residue or a nitrogenous base. In addition, the substituent (R₁) includes RNA chains of various structures containing natural and unnatural modifications of the terminal nucleotide region (5′ end), such as 5′-hydroxyl, phosphate, azide, amino and nucleoside groups (formulas 2a-2c), whose presence has consequences in the area of chemical and biological properties of the N-(2-aminoethyl)morpholine-based RNA analog. The nitrogenous base (R₂ according to formula 1) is a heterocyclic organic compound from the class of pyrimidines and purines, which together with a sugar residue forms a nucleoside residue by means of an N-glycosidic bond (as in the case of adenosine, guanosine, N6-methyladenosine, N7-methylguanosine, inosine, uridine, 5-methyluridine, cytidine and 5-methylcytidine) or C-glycosidic bond (as in the case of pseudouridine and 1-methylpseudouridine). A functional substituent (R₃ according to formula 1) is a functional group or motif, in particular a bioorthogonal group, a fluorophore, affinity tag or nucleic acid motif (according to formulas 3a-3e), connected to the N-(2 aminoethyl)morpholine group of an RNA analog by means of a linker (substituent X according to formulas 3a-3e). The presence of a functional group or motif is critical to the chemical, spectroscopic and biological properties of the N-(2-aminoethyl)morpholine-based RNA analog. The presence of a bioorthogonal functional group (according to formula 3a), such as azide, alkyne or amine, makes it possible to carry out a selective CuAAC reaction, SPAAC reaction, reaction with NHS ester or other reactions that do not involve chemical groups found in natural nucleic acids. The presence of a functional motif with the structure of a fluorophore, for example from the group of cyanines, rhodamines or fluoresceines (according to formulas 3b-3c), gives fluorescent properties that enable the use of N-(2 aminoethyl)morpholine-based RNA analog as a fluorescent probe, for example in microscopic observations or in studies of the activity of nucleolityc enzymes. The presence of a functional motif with an affinity tag structure such as biotin (according to formula 3d) allows the binding of the N-(2-aminoethyl)morpholine-based RNA analog by binding proteins such as streptavidin, avidin and their analogs, and the use of the RNA analog as an affinity probe. A functional motif with a nucleic acid structure (according to formula 3e) extends the polynucleotide sequence of the N-(2-aminoethyl)morpholine-based RNA analog present in the structure of the RNA chain with the oligonucleotide sequence of the motif. The linker (substituent X according to formulas 3a-3e) is a serial combination of simple chemical groups. The length and structure of the linker have minor impact on the properties of the functional group ormotif, as well as the properties of the N-(2-aminoethyl)morpholine RNA analog as a whole.

The method of obtaining N-(2-aminoethyl)morpholine-based RNA analogs is to subject a nucleic acid such as RNA (according to formula 4) to two subsequent chemical reactions. In step (i), the cis-diol group of the RNA is reacted with metaperiodic acid (or its salt), leading to the formation of a dialdehyde (according to formula 5). In step (ii), the resulting dialdehyde is further reacted with the ethylenediamine analog (according to formula 6) in a reducing medium, yielding the N-(2-aminoethyl)morpholine-based RNA analog. Both steps are carried out in an aqueous environment and can be carried out in one reactor. Furthermore, step (i) is performed efficiently and selectively using the following conditions: low periodate concentration (1.0-1.5 mM), wide range of RNA concentrations (1-100 μM), and at low temperature (below 40° C.). Step (ii) is optimal under the following conditions: in a reducing medium that allows selective reductive amination to proceed (for example in the presence of sodium cyanoborohydride at a concentration of 20-100 mM), in a buffered solution (pH in the range 5.5-7.5) and in the presence of an excess of the ethylenediamine analog (for example at a concentration of 1-10 mM). Step (ii) may be performed with the optional addition of an organic solvent to increase the solubility of the ethylenediamine analog (for example in a water:DMSO 4:1 v/v mixture). Optimal conditions for the preparation of N-(2-aminoethyl)morpholine-based RNA analogues are crucial for efficient synthesis with the use of an RNA substrate with a high degree of polymerization (above 30 nucleotides). The resulting N-(2-aminoethyl)morpholine-based RNA analog can be isolated from the reaction mixture by conventional methods such as chromatographic methods, nucleic acid salt precipitation, or commercially available nucleic acid isolation kits. The RNA substrate (according to formula 4) contains substituents (R₁ and R₂ according to formula 4) which are the precursors of two of the three main substituents of the N-(2-aminoethyl)morpholine-based RNA analog: the RNA chain (R₁ according to formula 1) and the nitrogenous base (R₂ according to formula 1). Therefore, the RNA chain and the nitrogenous bases of the substrate have analogous structure and properties to the RNA chain and the nitrogenous bases of the product, and the RNA substrate must contain at least one cis-diol moiety, for example as part of the ribose structure (formulas 4a-4c). The ethylenediamine analog (according to formula 6) contains a substituent (R₃ according to formula 6), which is a precursor of the functional group (R₃ according to formula 1)—one of the three main substituents of the N-(2-aminoethyl)morpholine moiety of the RNA analog. Therefore, the functional group of the ethylenediamine analog has an analogous structure and properties to the functional group of the RNA analog.

The analogs of ethylenediamine (according to formula 7) may be used as reagents in the synthesis of N-(2-aminoethyl)morpholine RNA analogs. They contain a reactive ethylenediamine motif (H₂N—(CH₂)₂—NH—CH₂—R) and a functional motif (such as a fluorophore, affinity tag or nucleic acid motif according to formulas 7a-7d) linked by a linker (substituent X according to formulas 7a-7d). The presence of the reactive ethylenediamine motif is crucial from the perspective of the rate and selectivity of the reactions taking place during step (ii) of the process for the preparation of N-(2-aminoethyl)morpholine-based RNA analog. Compounds containing structurally similar motifs, such as (H₂N—(CH₂)₂—NH—CO—R) or (H₂N—(CH₂)₃—NH—CH₂—R), do not have the properties necessary to efficiently obtain RNA analogs by the described method according to the invention. The functional motif of the ethylenediamine analog (according to formulas 7a-7d) is of key importance from the perspective of the chemical, spectroscopic and biological properties of the target N-(2-aminoethyl)morpholine-based RNA analog, but at the same time it cannot disrupt the reactivity of the ethylenediamine analog or any of the steps in the preparation of this RNA analog. The linker (substituent X according to formulas 7a-7d) is a serial combination of simple chemical groups, the length and structure of which is of marginal importance from the perspective of the properties of the ethylenediamine analog in general, as long as it does not interfere with the reactivity of the ethylenediamine analog or any of the steps in the preparation of the RNA analog. These types of ethylenediamine analogs can be obtained in single, selective and efficient synthetic step, such as the CuAAC reaction between an azide derivative of a functional motif and N-propargylethylenediamine or a reaction between a functional derivative of an NHS ester with diethylenetriamine.

### DETAILED DESCRIPTION OF THE INVENTION

In the course of research on the reactivity of the 3′ end of RNA, it was surprisingly found that the structure of the amine derivative used for the reaction with ribonucleotides or ribonucleic acids oxidized with periodate has a significant influence on the rate and efficiency of the process. Using mononucleotide (GMP) and trinucleotide (pU3) as RNA models, it was surprisingly found that selected ethylenediamine analogs (H₂N—(CH₂)₂—NH—CH₂—R) showed greater reactivity towards ribose dialdehyde derivatives than other amines and amine derivatives. Reactions with ethylenediamine and its analogs were faster and more efficient than analogous reactions with hydrazine and its analogs, as well as with a number of amines not containing the ethylenediamine motif (FIG. 2). A detailed analysis of the course of the reductive amination reaction and the intermediate products formed in it allowed for the formulation of its hypothetical mechanism, dependent on the amine used (FIG. 3). Based on the results of these preliminary studies, ethylene diamine analogs containing functional groups such as biotin (Biot-EDA), fluorescent dyes (FAM-EDA, pHrodo-EDA, Cy3-EDA, Cy5-EDA) or nucleotides (EDA-AG, EDA-m⁷Gp₃G) was carried out, in order to use them to modify RNA (FIG. 4). The synthesis of these analogs follows analogous synthetic pathways, for example by the reaction of the NHS ester with diethylenetriamine (synthesis of FAM-EDA and pHrodo-EDA) or the CuAAC reaction between N-propagylethylenediamine and azide (synthesis of Biot-EDA, Cy3-EDA, Cy5-EDA, EDA-AG and EDA-m⁷Gp₃G). Reactions of NHS esters or CuAAC are known for their high selectivity, efficiency and mild conditions. For this reason, there is a wide range of fluorescent dyes, affinity tags, amino acids, peptides, nucleosides, nucleotides, nucleic acids, nanoparticles and other substances containing azide, alkyne or NHS esters on the market. As a result, the proposed synthesis of ethylene diamine analogues is extremely simple, efficient and applicable to a wide range of derivatives. A number of experiments were conducted to understand the conditions under which the modification reaction of 3′ end of RNA proceeds efficiently and selectively with minimal RNA degradation. It was observed that the highest dialdehyde stability is achieved, regardless of the pH, below 40° C. and at the concentration of sodium cyanoborohydride not exceeding 100 mM. The reaction rate did not change significantly with a pH in the range of 5.5-7.5 and with an ethylenediamine analog concentration in the range of 1-10 mM. Further research was carried out on the stability and reactivity of RNA and on further optimization of the reaction conditions. The best results were obtained by carrying out a one-pot reaction, where RNA in a wide range of concentrations (1-100 μM) and length (3-2000 nt) was first incubated in the presence of NaIO₄ (1.0-1.5 mM) for 30 min at 25° C. without access to light. Subsequently, reagents (either separately or simultaneously, as a mixture), such as buffer (KH₂PO₄, pH 6, 100 mM), reducing agent (NaBH₃CN, 20 mM) and the ethylenediamine analog (1 mM) were added to the oxidized RNA solution. After incubation at 25° C. for 120 min, the N-(2-aminoethyl)morpholine-based RNA analog product could be efficiently isolated by simple methods, such as alcohol precipitation of RNA salt (80-90% isolation yield), using commercially available RNA isolation kits (isolation efficiency 90-100%) or by means of HPLC (isolation efficiency 25-60%).

The method according to the invention allows for direct, inexpensive (without the use of enzymes and catalysts) and quick modification of RNA with high efficiency: 75-99% for RNA with a length of 1-300 nucleotides and 65-80% for RNA with a length of 900-2100 nucleotides (FIG. 5A-C). RNA analogs can contain functional chemical moieties, such as fluorescent dyes, biotin or reactive bioorthogonal groups, such as amines, azides and alkynes.

In the preparation of RNA analogs, several chemical processes can be performed in parallel, including the SPAAC reaction, which was used to obtain RNA analogs containing two selectively placed fluorescent dyes to form a FRET pair (Cy3 and Cy5). For this purpose, enzymatic synthesis (in vitro transcription) of RNA was performed using T7 RNA polymerase and nucleotide analogs of substrates, thanks to which it was possible to obtain RNA containing an azide group within the structure of the 5′ cap of mRNA [8]. The transcription products were then used as substrates in a modified chemical labeling protocol, in which simultaneously the azide group at the 5′ end underwent SPAAC reaction and the dialdehyde at the 3′ end underwent reductive amination, leading to a doubly modified RNA derivative. The reaction products were purified by HPLC, which allowed for the isolation of RNA molecules containing both modifications (FIG. 5D-F). N-(2-aminoethyl)morpholine-based RNA analogs with a length of 35 and 276 nucleotides, labeled with Cy3 and Cy5 dyes (RNAS, RNA8, RNA14, Table 1) have been used as FRET probes to study RNA conformational changes and to monitor the activity of enzymes, such as RNase A, RNase T1, Dcp1/2, RNase R (FIG. 6) and RNase H (FIG. 7).

On the other hand, N-(2-aminoethyl)morpholine-based mRNA analogs encoding Gaussia luciferase and eGFP (enhanced green fluorescence protein) (993 and 1100 nucleotides in length, respectively) labeled with Cy3 and Cy5 dyes (RNA17-19 and RNA21-23, Table 1) were used in microscopic observations and in translation studies (FIG. 8). The latter show that the introduced modification of 3′ end of mRNA according to the invention does not disturb the protein biosynthesis process.

Another application of the method according to the invention is the chemical ligation of two RNA molecules. By means of in vitro transcription and the ethylene diamine analogs EDA AG and EDA m⁷Gp₃G, an RNA with the length of 35 nucleotides containing an ethylenediamine group at the 5′ end (RNA6, RNA9, Table 1) was obtained. RNA6 was used in a chemical labeling protocol modified according to the invention, in which this RNA was hybridized with complementary DNA (Table 3), allowing the 5′ and 3′ ends of two RNA molecules to be brought closer together, and then oxidized and subjected to an intermolecular reductive amination reaction. By means of polyacrylamide gel electrophoresis, the formation of RNA ligation products with lengths being a multiple of the substrate length was observed (FIG. 9).

### SHORT DESCRIPTION OF THE FIGURES

For a better understanding of the invention, it has been illustrated in the embodiments and in the attached tables and figures, in which:

FIG. 1 shows: A) General scheme for the modification of RNA by periodate oxidation and subsequent amination or reductive amination. R is a functional substituent, X is nitrogenous base, NA is nucleic acid; B) Structures of the amine derivatives used to modify RNA according to the prior art.

FIG. 2 shows the course of the reductive amination reaction according to the invention for the pUUU trinucleotide oxidized with NaIO₄, monitored by HPLC. A) Reaction scheme. B) Reaction yield as a function of time for methylamine (reference reaction), hydrazine (reference reaction), ethylenediamine (reference reaction), and ethylenediamine analogs (reaction according to the invention). C) The reaction rate constants determined assuming that the reaction is of a second-order.

FIG. 3 shows the course of the reductive amination reaction according to the invention for a GMP mononucleotide oxidized with NaIO₄, monitored by HPLC. A) Reaction course for ethylenediamine. B) Reaction course for hydrazine. C) Reaction course for cysteamine.

FIG. 4 shows the structures of the ethylenediamine analogs according to the invention obtained for modifying RNA.

FIG. 5 shows the fluorescent RNA labeling products according to the invention. A-C) HPLC chromatograms of labeling of 3′ end of RNA with the length of A) 35 (RNA1 substrate, RNA2 product), B) 237 (RNA10 substrate, RNA11 product), or C) 2098 (RNA24 substrate, RNA25 product) nucleotides with Cy3 fluorescent dye (S is the unreacted starting material, P is the reaction product). D-F) HPLC chromatograms of labeling of 5′ and 3′ end of RNA with the length of D) 35 (RNA3 substrate, RNAS product), E) 276 (RNA12 substrate, RNA14 product), F) or 993 (RNA16 substrate, RNA19 product) nucleotides with dyes Cy5 and Cy3, respectively (S is unreacted substrate, P is the major reaction product, and 3 and 5 are intermediates, mono-labeled at the 3′ or 5′ end, respectively).

FIG. 6 shows the monitoring of the progress of enzymatic reactions with FRET probes. Changes in the fluorescence spectrum of RNA5 (A-D) and RNA8 (E) labeled with Cy5 and Cy3 dyes at the 5′ and 3′ ends, respectively, in the presence of enzymes with nucleolytic activity. Ribolock—a commercially available, selective RNase A inhibitor. F) The ratio of the fluorescence intensity at 564 and 667 nm as the enzymatic reaction progresses.

FIG. 7 shows the monitoring of RNase H activity with FRET probes. A-D) Changes in the RNA5 fluorescence spectrum without and in the presence of RNase H and different DNAs with sequences complementary to the probe sequence. E) The ratio of the fluorescence intensity at 564 and 667 nm over time.

FIG. 8 shows the microscopic observations and expression of genes encoded by fluorescent mRNA analogs in HeLa cells. A) Time dependence of Gaussia luciferase activity (luminescence) after mRNA (RNA16-19) transfection and B) total relative activity of the protein after 88 h of incubation (averaged two biological replicates). C) Measurements of the fluorescence intensity of eGFP protein and Cy3 and Cy5 dyes in cells, after transfection of fluorescent mRNA analogs encoding the GFP protein (RNA20-23), performed by flow cytometry. D) Confocal microscopy images of cells after transfection with fluorescent mRNA analogs. mock-test without mRNA; ppp-Ggluc-test with translationally inactive mRNA (RNA15); N₃-m⁷Ggluc and N₃-m⁷Gegfp-tests with unlabeled mRNA (RNA16 and RNA20); N₃-m⁷Ggluc-Cy3 and N₃-m⁷Gegfp-Cy3-tests with mRNA labeled with Cy3 at 3′ end using method according to the invention (RNA17 and RNA21); N₃-m⁷Ggluc-Cy3 mock-test with mRNA labeled with Cy3 at 3′ end using method according to the invention (RNA17), with omitting the NaIO₄ oxidation step; Cy5-m⁷Ggluc and Cy5-m⁷Gegfp-tests with mRNA labeled at 5′ end (RNA18 and RNA22); Cy5-m7Ggluc-Cy3 and Cy5-m⁷Gegfp-Cy3-tests with mRNA labeled at 5′ and 3′ end (RNA19 and RNA23), including labeling at 3′ end performed according to the invention.

FIG. 9 shows the chemical ligation of RNA6. A) Reaction scheme. B) Polyacrylamide gel containing substrate (NR) and reaction products in the presence of DNA (A0-D33, Table 3) or without DNA (H₂O).

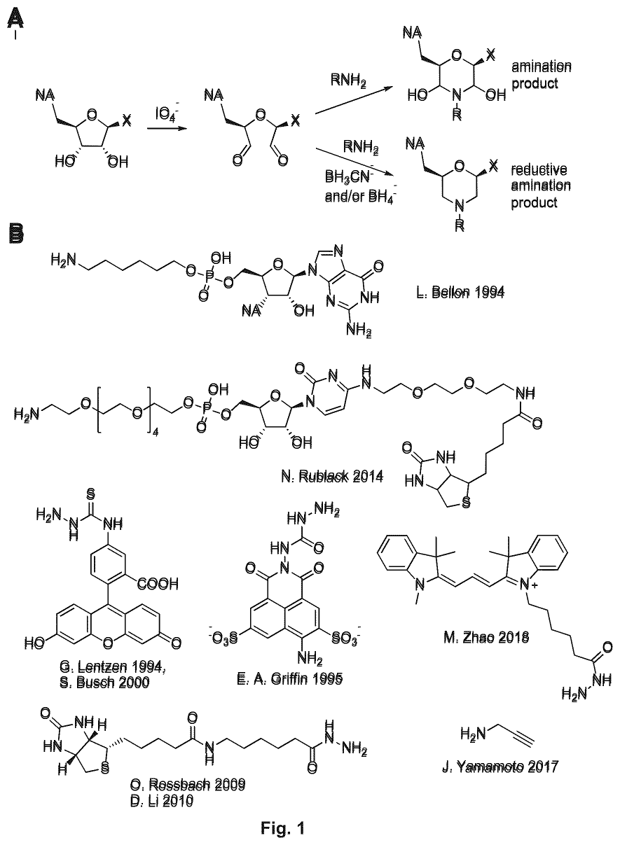

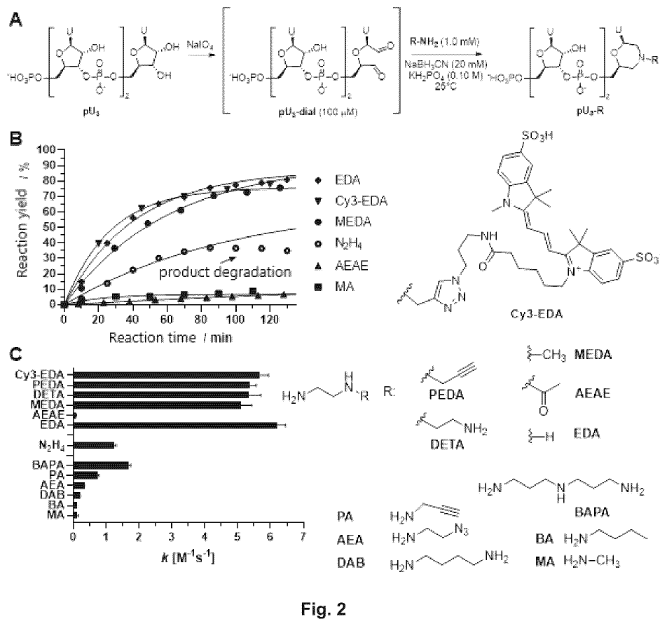

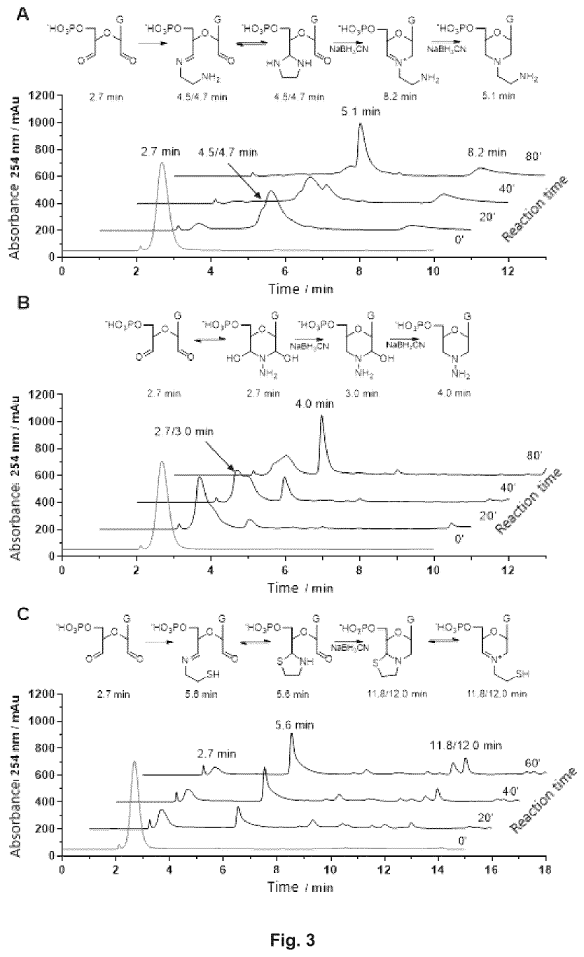

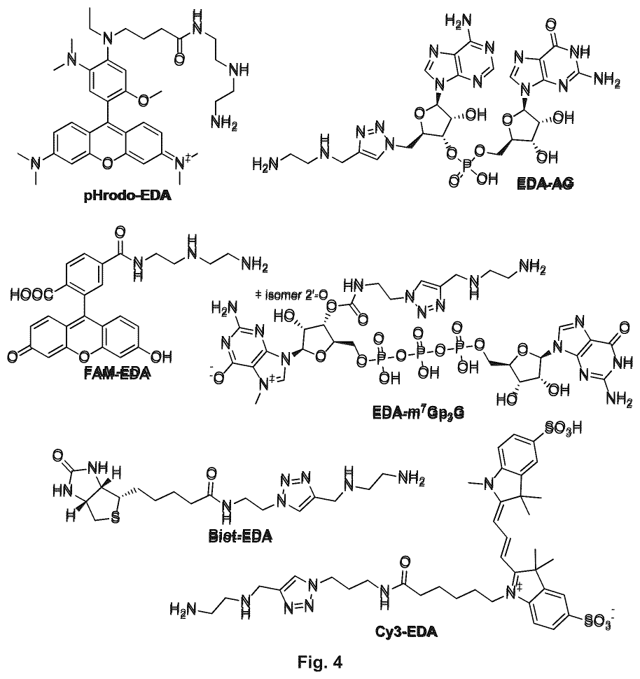

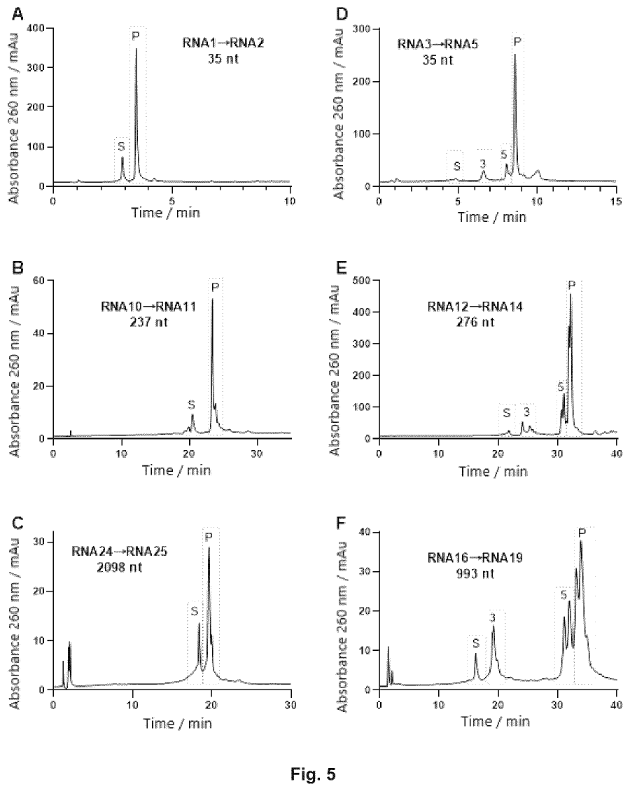

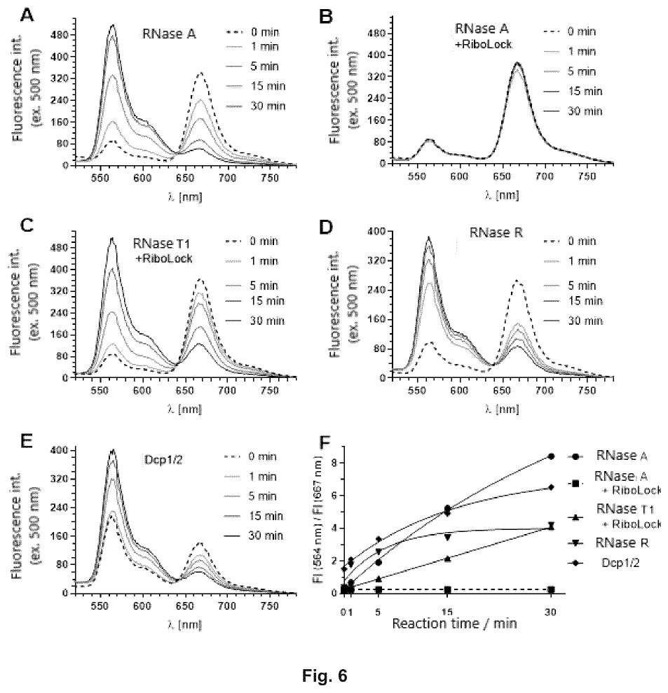

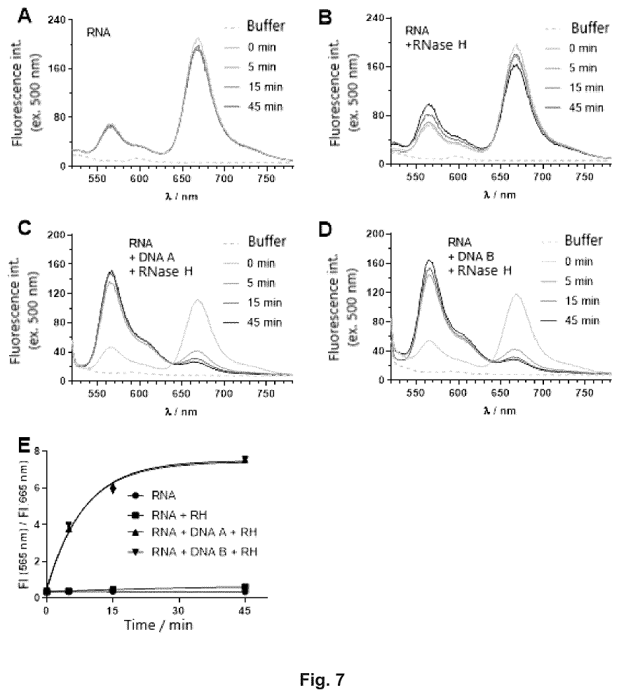

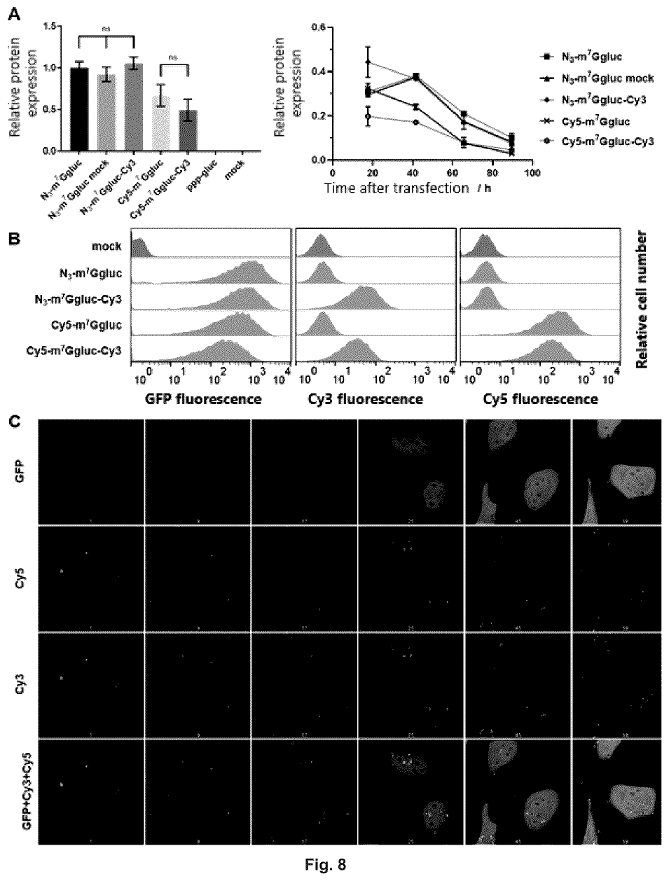

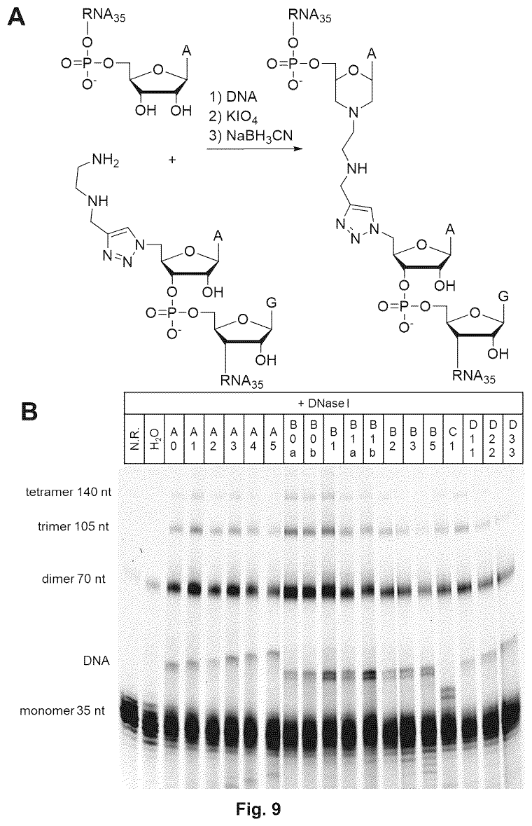

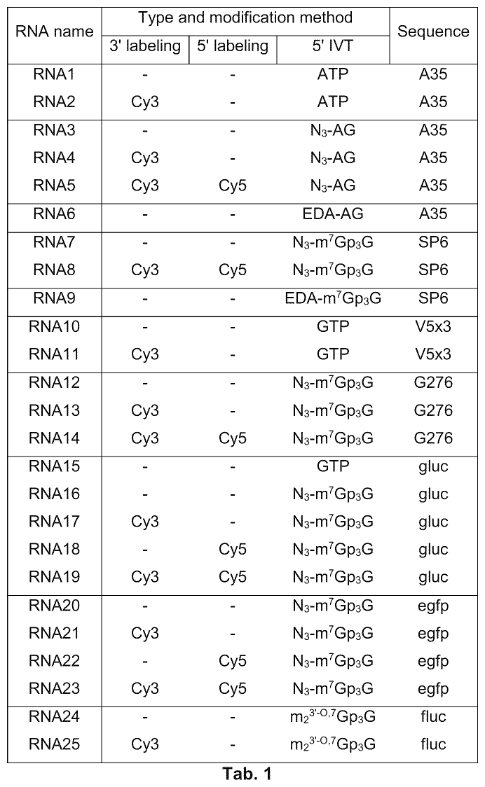

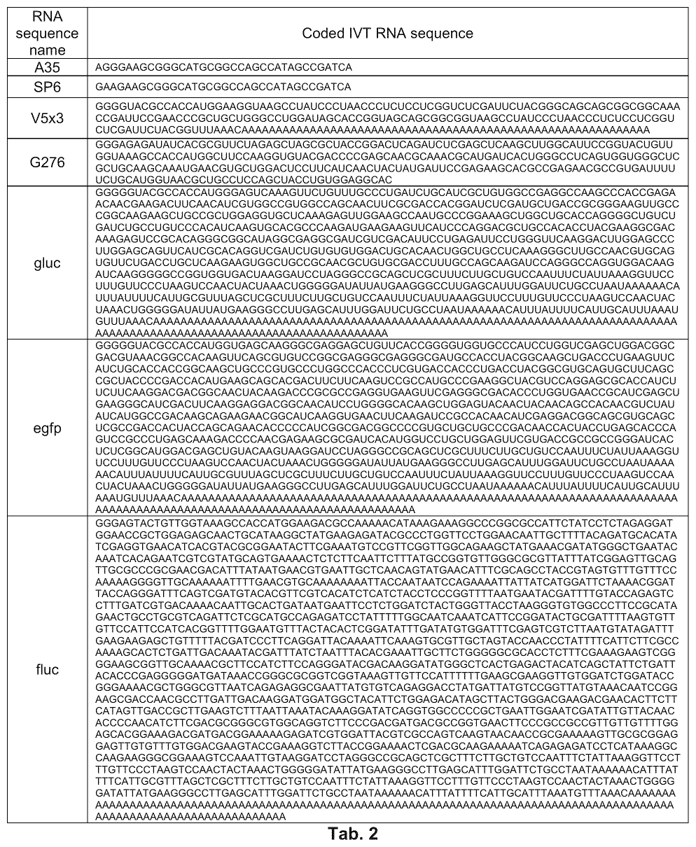

Table 1 shows the names of the obtained RNAs and type and modification methods thereof: 5′ IVT is a nucleotide or its analog introduced at the 5′ end of RNA during the transcription reaction; 3′ labeling: means that RNA of interest was subjected to a 3′ end labeling reaction with Cy3 according to the invention; 5′ labeling: means that the RNA of interest was subjected to a 5′ end labeling reaction with Cy5. Double labeling products (with 3′ Cy3 and 5′ Cy5 simultaneously) contain both Cy3 and Cy5;

Table 2 shows the RNA nucleotide sequences of Table 1.

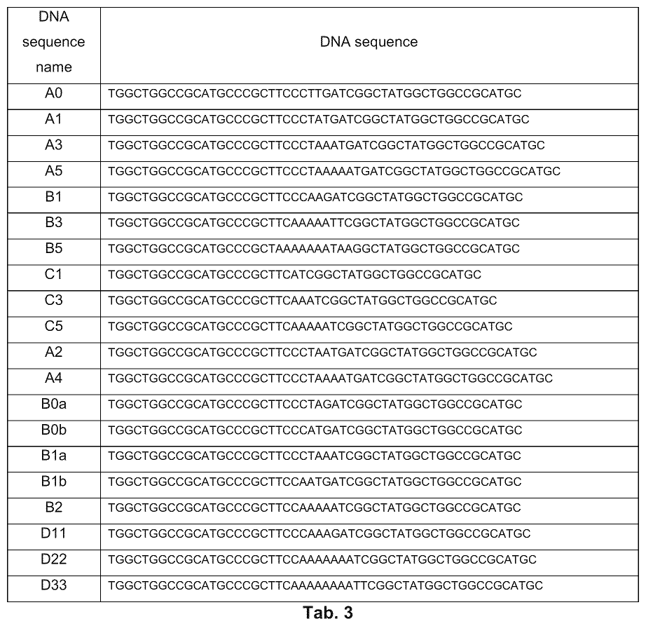

Table 3 shows the DNA sequences used during the chemical ligation of RNA6 (FIG. 9)

### EXAMPLES

The following examples are provided only to illustrate the invention and to explain its particular aspects, not to limit it, and should not be construed as falling within its entire scope as defined in the appended claims. The following examples used standard materials and methods employed in the art or followed manufacturers' recommendations for specific materials and methods unless otherwise indicated.

### Synthesis of tert-butyl (2-bromoethyl)carbamate

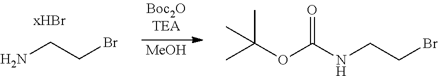

The synthesis was carried out based on the published protocol [23]. Triethylamine (1.38 mL, 9.92 mmol) was added to a solution of 2-bomoethylamine hydrobromide (1.02 g, 4.96 mmol) in methanol (70 mL) at 0/4° C. Then, a solution of di-tert-butyl dicarbonate (1.09 g, 4.99 mmol) in methanol (20 mL) was added. After stirring for 15 min at 0/4° C., the cooling bath was removed and the solution was stirred for 2 h at room temperature. The solution was diluted with water/dichloromethane (65 mL/65 mL), transferred to a separatory funnel and washed with dichloromethane (65 mL). The combined organic fractions were dried over Na₂SO₄, filtered and concentrated under reduced pressure. The product was obtained in the form of a colorless oil (1.11 g, 4.95 mmol, ˜100%). ¹H NMR (500 MHz, CDCl₃) σ [ppm]: 4.94 (br s, 1H, CONH), 3.54 (t, J=5.8 Hz, 2H, CH₂CH₂), 3.45 (t, J=5.8 Hz, 2H, CH₂CH₂), 1.45 (s, 9H, tBu).

### Synthesis of tert-butyl (N-propargylaminoethyl)carbamate

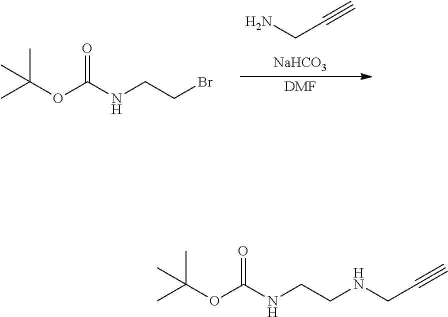

Sodium bicarbonate (200 mg, 2.38 mmol), DMF (2.0 mL), and propargylamine (849 μL, 13.25 mmol) were added to tert-butyl (2-bromoethyl)carbamate (495 mg, 2.21 mmol). The suspension was refluxed at 60° C. for 4.5 h. The course of the reaction was monitored on TLC (n-hexane/2-propanol 1:1, ninhydrin staining, RF˜0.4). The suspension was diluted with a mixture of saturated sodium carbonate and dichloromethane (20 mL/30 mL), transferred to a separatory funnel and washed with dichloromethane (2×20 mL). The combined organic phases were dried with Na₂SO₄, filtered and concentrated under reduced pressure. The obtained ginger oil was separated by FLASH chromatography (dryload, 12 g silicagel cartridge) with a step gradient of 2-propanol in n-hexane. The fractions containing the desired product were combined and concentrated under reduced pressure. The product was obtained in the form of a yellow oil (374 mg, 1.88 mmol, 86%). ¹H NMR (500 MHz, CDCl₃) δ [ppm]: 3.42 (d, J=2.4 Hz, 2H, CH₂CCH), 3.24 (q, J=5.7 Hz, 2H, CH₂CH₂), 2.81 (t, J=5.7 Hz, 2H, CH₂CH₂), 2.22 (t, J=2.4 Hz, 1H, CCH), 1.44 (s, 9H, tBu).

### Synthesis of N-Propargylethylenediamine (PEDA) Dihydrochloride

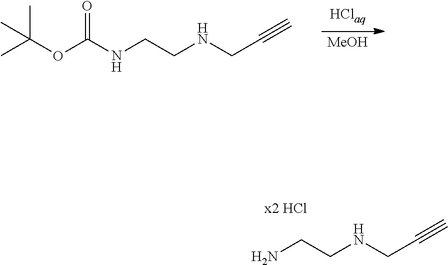

Hydrochloric acid (4 mL, ˜37 wt %) was added dropwise to a solution of tert-butyl (N-propargylaminoethyl)carbamate (374 mg, 1.88 mmol) in methanol (20 mL). After 30 min of incubation at room temperature, ethanol was added and the solution was evaporated under reduced pressure. Anhydrous ethanol was then added portionwise until precipitation occurred. Then the mixture was cooled, the precipitate was filtered and washed with a minimum volume of cold anhydrous ethanol (a total of 100 mL of anhydrous ethanol was consumed). The precipitate was dissolved in water and lyophilized to obtain the product as a light-brown powder (156 mg, 0.912 mmol, 48%). ¹H NMR (500 MHz, D₂O) δ [ppm]: 4.08 (d, J=2.6 Hz, 2H, CH₂CCH), 3.58 (td, J=7.0, 1.8 Hz, 2H, CH₂CH₂), 3.46 (td, J=7.0, 1.9 Hz, 2H, CH₂CH₂), 3.10 (t, J=2.6 Hz, 1H, CCH). ¹³NMR (126 MHz, D₂O) δ [ppm]: 79.01 (CCH), 43.11 (CH₂CH₂), 36.98 (CH₂CCH), 35.37 (CH₂CH₂).

### Synthesis of 2-azidoethylamine

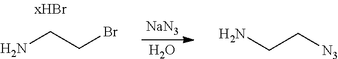

The synthesis was carried out based on the published protocol [8]. 2-Bromoethylamine hydrobromide (10.09 g, 49.3 mmol) was added to a solution of sodium azide (8.14 g, 148 mmol) in water (40 mL) and refluxed at 70° C. for 16 h. The mixture was cooled and a solution of potassium hydroxide (14 g) in water (10 mL) was added, followed by dichloromethane (50 mL). After stirring at room temperature for 30 min, the suspension was transferred to a separatory funnel and washed with dichloromethane (5×50 mL). The combined organic phases were dried with Na₂SO₄, filtered and carefully concentrated under reduced pressure (in the pressure range of 600-50 mbar at 30° C., until the weight of the product was stabilized). The product was obtained in the form of a colorless oil (3.78 g, 43.9 mmol, d=1.04 g/ml, 89%).

### Synthesis of tert-butyl [N-(2-azidoethyl)aminoethyl]carbamate

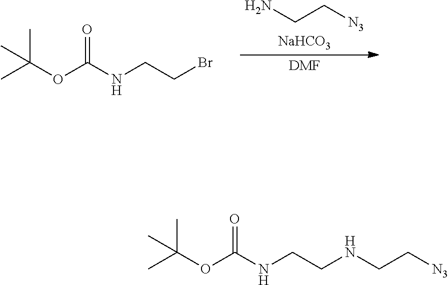

A solution of tert-butyl (2-bromoethyl)carbamate (1.47 g, 6.58 mmol) in DMF (2 mL) was added dropwise to a suspension consisting of 2-azidomethylamine (1.63 mL, 19.73 mmol), sodium bicarbonate (0.91 g, 6.58 mmol) and DMF (8 mL) for 1 h at 70° C. The course of the reaction was monitored by TLC (n-hexane/2-propanol 1:1, ninhydrin staining, RF˜0.4). The suspension was diluted with a mixture of saturated sodium carbonate and dichloromethane (40 mL/50 mL), transferred to a separatory funnel and washed with dichloromethane (3×50 mL). The combined organic phases were dried with Na₂SO₄, filtered and concentrated under reduced pressure. The resulting colorless oil was separated by FLASH chromatography (dryload, 12 g silicagel cartridge) with step gradient of 2-propanol in n-hexane. The fractions containing the desired product were combined and concentrated under reduced pressure. The product was obtained in the form of a colorless oil (940 mg, 4.14 mmol, 63%).

### Synthesis of N-(2-azidoethyl)ethylenediamine (AEEDA) dihydrochloride

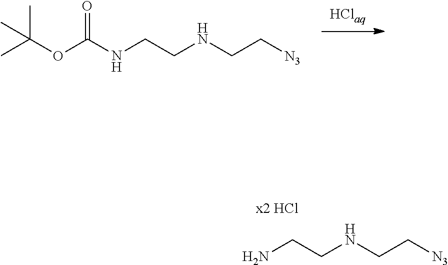 [SMILES: CC(C)(C)OC(=O)NCCNCCN=[N+]=[N-] ; [N-]=[N+]=NCCNCCN]

Dilute hydrochloric acid (5 mL, ˜10 wt %) was added dropwise to tert-butyl [N-(2-azidoethyl)aminoethyl]carbamate (374 mg, 1.88 mmol). After 1 h incubation at room temperature, the mixture was diluted with water (20 mL), transferred to a separatory funnel and washed with dichloromethane (3×25 mL). Ethanol was added to the aqueous phase and the solution was evaporated under reduced pressure. Anhydrous acetonitrile was then added portionwise until precipitation occurred. Then the mixture was cooled, the precipitate was filtered and washed with a minimum volume of cold anhydrous ethanol. The precipitate was dried under reduced pressure to obtain the product as a white powder (70 mg, 0.53 mmol, 13%). ¹H NMR (500 MHz, D₂O) σ [ppm]: 3.82 (t, J=5.5 Hz, 2H), 3.49 (m, 2H), 3.43 (m, 2H), 3.35 (t, J=5.5 Hz, 2H). ¹³C NMR (126 MHz, D₂O) σ [ppm]: 49.61, 49.48, 46.84, 38.01.

### Synthesis of Biot-EDA

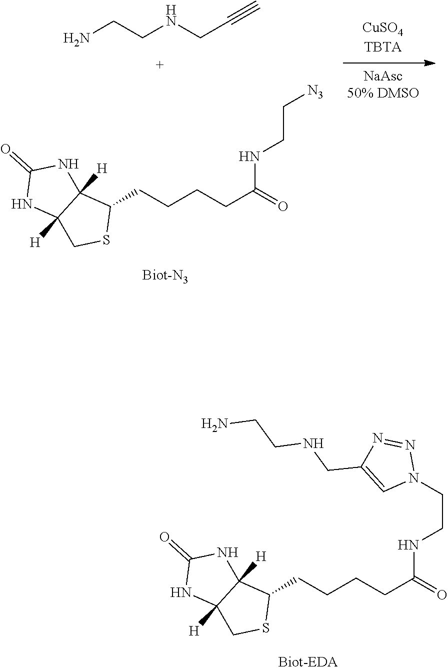

The synthesis of Biot-N₃ was carried out according to the known method [24]. A mixture of 50% DMSO (1.26 mL) and a solution of the CuSO4-TBTA complex (340 μL, 9.4/10 mM in 50% DMSO) was added to the weighed reagents Biot-N₃ (9.81 mg, 31.4 μmol), PEDA×2HCl (8.27 mg, 48.4 μmol) and sodium ascorbate (146 mg, 234 μmol). After being stirred for 105 min at room temperature, EDTA (400 μL of 0.5 M) and water (6 mL) were added to the solution. After filtration with a syringe filter, HPLC separation was carried out: SUPELCOSIL™ LC-18-T column, 250×4.6 mm, 5 μm, A—50 mM NH₄OAc pH 5.9, B—50 mM NH₄OAc pH 5.9/MeOH 1:1, 1.3 mL/min @22° C., programs: 0-100% B in 30 min (RT=15 min). After combining the fractions and freeze-drying three times, the Biot-EDA product was obtained in the form of the acetate salt (4.5 mg, 8.48 μmol, M=530.7 g/mol) with a yield of 29%. MS ESI(+): 411.4 (Calc. [M+H]⁺: 411.2).

### Synthesis of Cy3-EDA

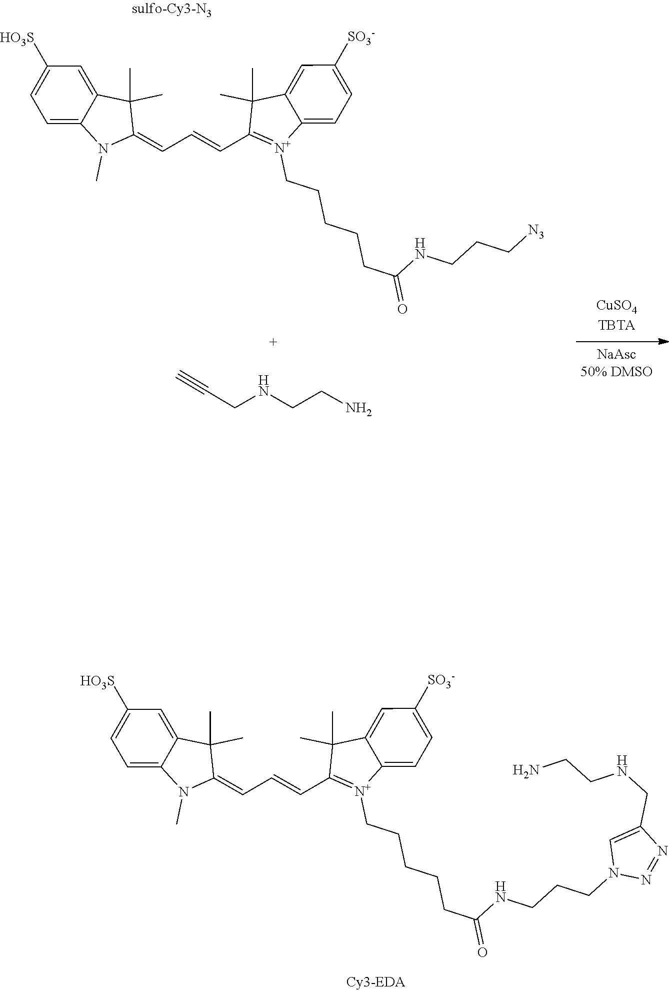

Sodium ascorbate (5.19 mg, 26.2 μmol) and PEDA×2HCl (1.13 mg, 6.63 μmol) were added to sulfo-Cy3-N₃ (2.22 mg, 3.01 μmol, Lumiprobe) and dissolved in 50% DMSO (270 μL). Then a solution of the CuSO₄-TBTA complex (32 μL, 9.4/10 mM in 50% DMSO) was added and incubated for 60 min at room temperature (22° C.). Then water (2.7 mL) and EDTA solution (6 μL, 0.5 M, pH 8.0) were added and HPLC separation was performed on a Gemini® NX-C18 column, 5 μm, 110 Å, 250×10 mm; system A: 100 mM TEAA pH 7.0 B: 75% MeCN, program: 0-27% B in 40 min, 100% B for 10 min, 5 mL/min @25° C. (RT=35 min). After combining the fractions and freeze-drying three times, and dissolving in 50% DMSO (180 μL), the product was obtained in the form of a triethylamine acetate salt solution (1.66 μmol, 9.2 mM, A₅₅₀=1492, ε₅₄₈=162 mM⁻¹cm⁻¹) with a 55% yield. MS ESI(−): 795.6 (Calc. [M−H]⁻: 796.3) ESI(+): 797.4 (Calc. [M−H]⁻: 797.3).

### Synthesis of Cy5-EDA

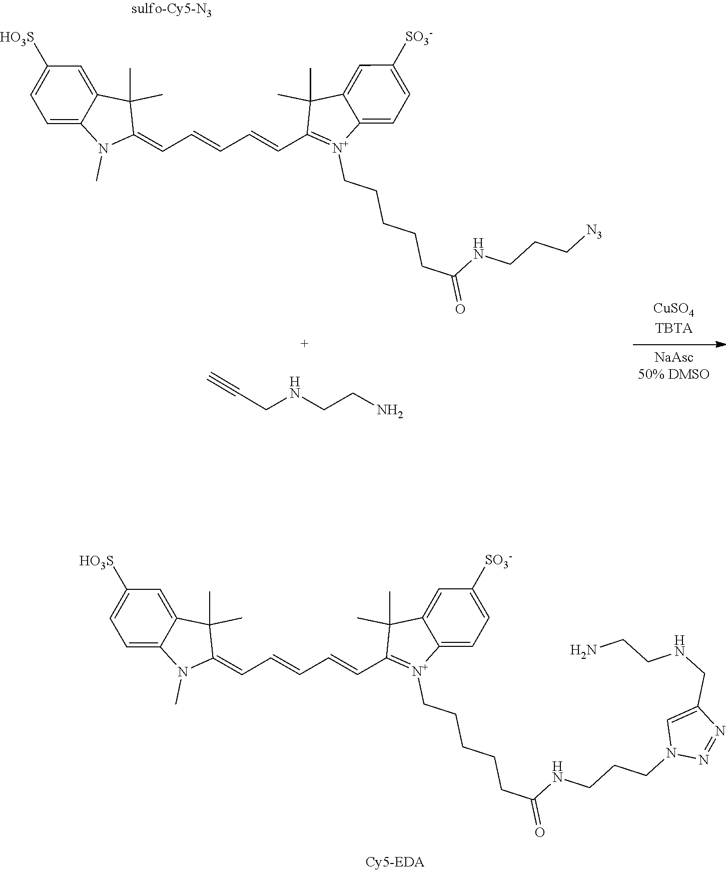

The synthesis was carried out according to a protocol analogous to the synthesis of Cy3-EDA, starting from sulfo-Cy5-N₃ (4.7 mg, 6.01 μmol, Lumiprobe). After combining the HPLC fractions, freeze-drying three times and dissolving in water (360 μl), the product was obtained in the form of a triethylamine acetate salt solution (3.58 μmol, 9.8 mM, A₆₅₄=2450, ε₆₄₅=250 mM⁻¹cm⁻¹) with a yield of 59%. MS ESI(−): 821.8 (Calc. [M−H]⁻: 821.4) ESI(+): 823.5 (Calc. [M−H]⁻: 823.4).

### Synthesis of FAM-EDA

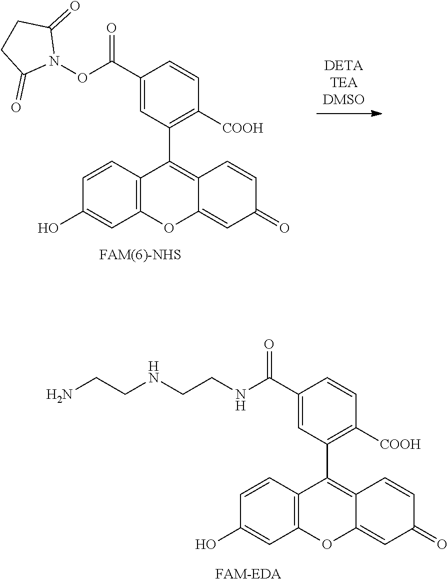 [SMILES: O=C(ON1C(=O)CCC1=O)c1ccc(C(=O)O)c(-c2c3ccc(=O)cc-3oc3cc(O)ccc23)c1 ; NCCNCCNC(=O)c1ccc(C(=O)O)c(-c2c3ccc(=O)cc-3oc3cc(O)ccc23)c1]

Triethylamine (10 μL, bioultra) and diethylenetriamine (30 μL) were added to a solution of NHS 6-carboxyfluorescein (4.5 mg, 9.5 μmol, ChemGenes) in DMSO (200 μL) and incubated at 22° C. for 60 min. Then an ethanol solution (1 mL, 80%) was added and evaporated under reduced pressure. This operation was repeated twice. Ammonium acetate solution (1.8 mL, 0.5 M, pH 5.9) was added and separation was performed by HPLC: Gemini® 5 μm NX-C18 column, 110 Å, 250×10 mm; system A: 50 mM NH₄OAc pH 5.9, B: MeOH; 0-50% B in 30 min, 5.0 mL/min @22° C. (RT=27 min). After freeze-drying three times, the product in the form of the ammonium acetate salt (2.8 μmol, ε₄₉₀=83 mM⁻¹cm⁻¹, 30%) was dissolved in 60% DMSO to give a 10 mM solution. MS ESI(−): 460.5 (Calc. [M−H]⁻: 460.2).

### Synthesis of pHrodo-EDA

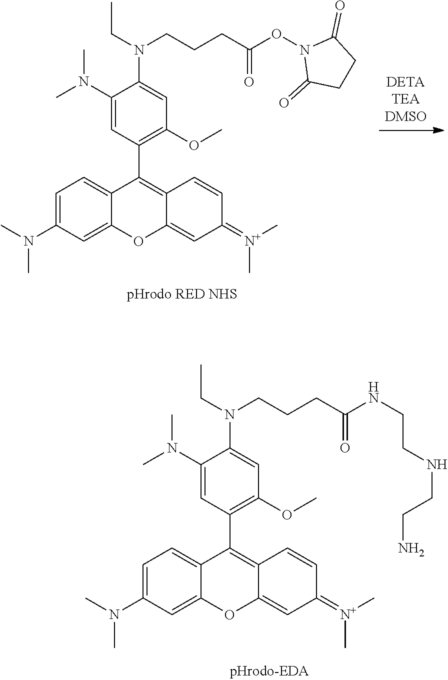 [SMILES: CCN(CCCC(=O)ON1C(=O)CCC1=O)c1cc(OC)c(-c2c3ccc(=[N+](C)C)cc-3oc3cc(N(C)C)ccc23)cc1N(C)C ; CCN(CCCC(=O)NCCNCCN)c1cc(OC)c(-c2c3ccc(=[N+](C)C)cc-3oc3cc(N(C)C)ccc23)cc1N(C)C]

Triethylamine (10 μL, bioultra) and diethylenetriamine (20 μL) were added to a solution of pHrodo RED NHS ester (1 mg, 2.0 μmol, Thermo) in DMSO (200 μL) and incubated at 22° C. for 60 min in the dark. Then an ethanol solution (1 mL, 80%) was added and evaporated under reduced pressure. This operation was performed twice. A solution of triethylamine acetate (1.8 mL, 0.1 M, pH 7.0) was added and separation was performed by HPLC: Gemini® 5 μm NX-C18 column, 110 Å, 250×10 mm; system A: 50 mM AA pH 5.9, B: MeCN; 0-100% B in 60 min, 5.0 mL/min @22° C. (RT=26 min, MS). After double lyophilization and dissolving in water (100 μL), a product solution (2.8 μmol, ε₅₆₀=65.0 mM⁻¹cm⁻¹) was obtained, with a concentration of 12.6 mM (A₅₆₀=82, A₂₆₀=30, 1 mm, pH 7.0) with a yield of 63%. MS ESI(+): 630.4 (Calc. [M−H]⁻: 630.4).

### Synthesis of EDA-m⁷Gp₃G

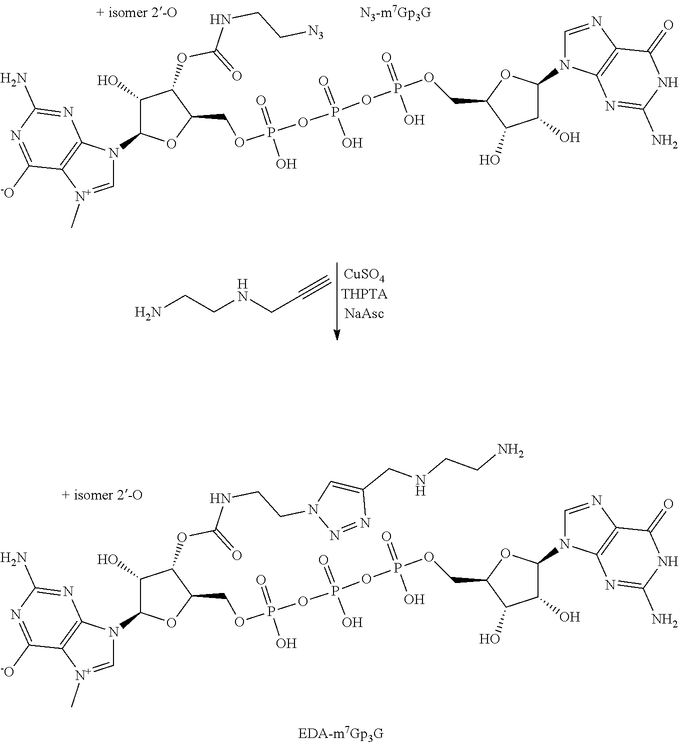

Sodium ascorbate (10.3 mg, 52 μmol) and PEDA×2HCl (3.4 mg, 20 μmol) were added to N₃-m⁷Gp₃G [8] (10 mg, 8.4 μmol) and dissolved in a degassed triethylamine acetate solution (2.0 mL, 45 mM, pH 7). Then a solution of the CuSO₄-THPTA complex (10 μL, 100/500 mM in water) was added and incubated for 2 h at room temperature. Then, an EDTA solution (20 μL, 100 mM, pH 7.0) was added and HPLC separation was performed on a C18 column; system A: 100 mM NH₄OAc pH 5.9, B: 30% MeCN, program: 0-25% B in 40 min, 4.5 mL/min @22° C. After combining the fractions and freeze-drying three times, the product was obtained in the form of the ammonium acetate salt (1.6 mg, 1.75 μmol, ε₂₆₀=20.0 mM⁻¹cm⁻¹) with a yield of 21%. MS ESI(−): 505.4 (Calc. [M−2H]²⁻: 505.1), ESI(+): 507.4 (Calc. [M+2H]²⁺: 507.1).

### Synthesis of pU₃

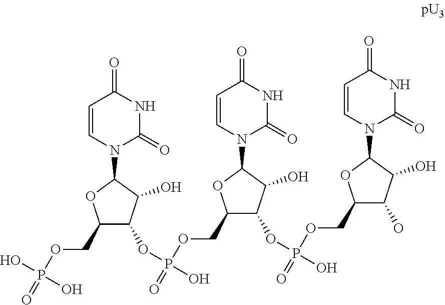 [SMILES: O=c1ccn([C@@H]2O[C@H](COP(=O)(O)O[C@H]3[C@@H](O)[C@H](n4ccc(=O)[nH]c4=O)O[C@@H]3COP(=O)(O)O[C@H]3[C@@H](O)[C@H](n4ccc(=O)[nH]c4=O)O[C@@H]3COP(=O)(O)O)[C@@H](O)[C@H]2O)c(=O)[nH]1]

The synthesis was carried out on the basis of the known method using the AKTA Oligopilot plus 10 synthesizer on 5′-O-DMT-2′-O-TBDMS-rU 3′-|caa Primer Support 5G ribo U 300 (170 mg, 50.7 μmol, 298 μmol/g, GE Healthcare) solid support. During the coupling, the column solid support was washed with a solution of 5′-O-DMT-2′-O-TBDMS uridine phosphoramidite (ChemGenes) or biscyanoethyl phosphoramidite (ChemGenes) in acetonitrile (0.6 mL, 0.2 M, 2.4 eq) along with a solution of 5-(benzylthio)-1H-tetrazole in acetonitrile (0.30 M) for 15 min. A solution of dichloroacetic acid in toluene (3% v/v) was used as a detritilation reagent, an iodine solution in pyridine (0.05 M) was used as an oxidant, N-methylimidazole in acetonitrile (20% v/v) was used as Cap A and a mixture of acetic anhydride (40% v/v) and pyridine (40% v/v) in acetonitrile was used as Cap B. After the final synthetic cycle, the RNA product on the solid support was incubated in a solution of diethylamine in acetonitrile to remove 2-cyanoethyl groups. The solid support was washed with acetonitrile and dried with argon. For cleavage and deprotection of the product, the resin was incubated in AMA (3 mL of 40 wt % methylamine and 3 ml of 30 wt % ammonia water) for one hour at 40° C. The resulting solution was evaporated and the product was dissolved in DMAO (0.220 mL). TBDMS groups were removed with triethylamine trihydrofluoride (250 μL, 65° C., 3 h). After cooling, the solution was diluted with sodium bicarbonate solution (20 mL, 0.25 M). The product was isolated by ion exchange chromatography on DEAE Sephadex (0-1.2 M TEAB gradient). After evaporation of the fractions, the product was obtained in the form of the triethylammonium salt (29 mg, 21.0 μmol, 630 mOD₂₆₀, HPLC₂₆₀=99%, 57% yield). ESI(+): 467.1, 935.5 (Calc. [M+2H]²⁺: 469.1, [M+H]⁺: 937.1).

### Synthesis of N₃-AG

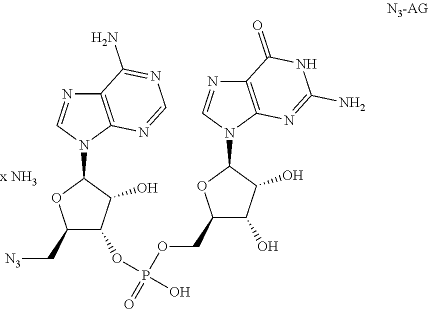 [SMILES: [N-]=[N+]=NC[C@H]1O[C@@H](n2cnc3c(N)ncnc32)[C@H](O)[C@@H]1OP(=O)(O)OC[C@H]1O[C@@H](n2cnc3c(=O)[nH]c(N)nc32)[C@H](O)[C@@H]1O]

The synthesis was carried out on the basis of the known method using the AKTA Oligopilot plus 10 synthesizer on 5′-O-DMT-2′-O-TBDMS-rG^{iBu} 3′-Icaa Primer Support 5G ribo G 300 (163 mg, 49.0 μmol, 300 μmol/g, GE Healthcare) solid support. During the coupling, the column solid support was washed with a solution of 5′-O-DMT-2′-O-TBDMS rA^{Pac} in acetonitrile (0.6 mL, 0.2 M, 2.4 eq) along with a solution of 5-(benzylthio)-1H-tetrazole in acetonitrile (0.30 M) for 15 min. A solution of dichloroacetic acid in toluene (3% v/v) was used as a detritilation reagent, an iodine solution in pyridine (0.05 M) was used as an oxidant, N-methylimidazole in acetonitrile (20% v/v) was used as Cap A and a mixture of acetic anhydride (40% v/v) and pyridine (40% v/v) in acetonitrile was used as Cap B. After the final synthetic cycle, the RNA product on the solid support was incubated in a solution of diethylamine in acetonitrile to remove 2-cyanoethyl groups. The solid support was washed with acetonitrile and dried with argon. The solid support was washed in a closed circuit with a solution of triphenoxymethylphosphine iodide ((PhO)₃PCH₃⁺I⁻) in DMF (1.0 mL, 0.6 M) for 15 min. The solid support was washed successively with DMF, acetonitrile, dried with argon and transferred to a test tube. A saturated solution of sodium azide in DMF (1 mL) was then added and vigorously stirred for one hour at 60° C. The solid support was washed successively with water, ethanol, acetonitrile, and dried with argon. For cleavage and deprotection of the product, the solid support was incubated in AMA (3 mL of 40 wt % methylamine and 3 mL of 30 wt % ammonia water) for one hour at 50° C. The resulting solution was evaporated and the product was dissolved in water. The product was isolated by ion exchange chromatography on DEAE Sephadex (gradient elution 0-0.6 M TEAB). After evaporation of the fractions, the product was obtained in the form of the triethylammonium salt (21 mg, 20.8 μmol, 550 mOD₂₆₀, E₂₆₀=24.3 mM⁻¹cm⁻¹ HPLC₂₆₀=92%, 42% yield).

For in vitro transcription, a portion of the product (11 mg, 11.1 μmol) was further purified by HPLC: Vydac Denali HiChrom C18 column, 150×10 mm, 5 μm, 120 Å; solvents A—50 mM NH₄OAc pH 5.9, B—50 mM NH₄OAc pH 5.9/MeCN 7:3 v/v, program: 0-25% B in 40 min, flow 4.5 mL/min at 25° C. (RT=25 min). After combining the fractions and freeze-drying three times, the product was obtained in the form of the ammonium salt (5.5 mg, 9.0 μmol, 220 mOD₂₆₀, E₂₆₀=24.3 mM⁻¹cm⁻¹, HPLC₂₆₀˜100%, 81% yield). MS ESI(−): 636.3 (Calc. [M−H]⁻¹: 636.1).

### Synthpgig of FnA-An

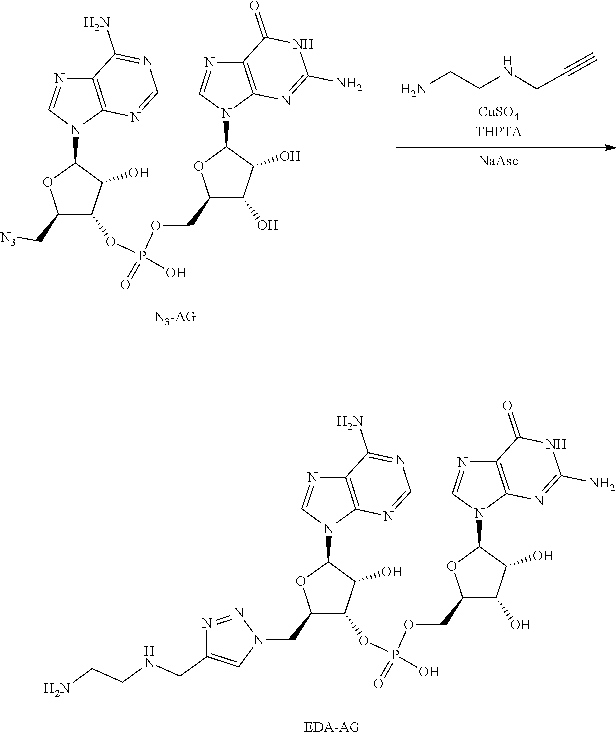

Sodium ascorbate (10.3 mg, 52 μmol) and PEDA×2HCl (3.4 mg, 20 μmol) were added to N₃-AG (7.4 mg, 11.5 μmol) and dissolved in degassed triethylamine acetate solution (2.0 mL, 45 mM, pH 7). Then a solution of the CuSO4-THPTA complex (10 μL, 100/500 mM in water) was added and incubated for 2 h at room temperature. Then an EDTA solution (20 μL, 100 mM, pH 7.0) was added and HPLC separation was performed on a C18 column; system A: 100 mM NH₄OAc pH 5.9 B: 30% MeCN, program: 0-25% B in 40 min, 4.5 mL/min @22° C. After combining the fractions and freeze-drying three times, the product was obtained in the form of the ammonium acetate salt (2.8 mg, 3.50 μmol, E₂₆₀=24.3 mM⁻¹cm⁻¹) with a yield of 30%. MS ESI(−): 734.4 (Calc. [M−H]⁻: 734.2).

### Preparation of Template DNA for In Vitro Transcription Reaction of RNA Having A35 and SP6 Sequences

RNA transcription template having A35 sequence (RNA1-6) was prepared as follows: solutions of two DNA oligonucleotides (Genomed) having sequences:

CAGTAATACGACTCACTATTAGGGAAGC
GGGCATGCGGCCAGCCATAGCCGATCA
(coding strand A35);
TGATCGGCTATGGCTGGCCGCATGCCCG
CTTCCCTAATAGTGAGTCGTATTACTG
(template strand A35);

were mixed 1:1 in hybridization buffer (4 mM Tris-HCl pH 7.5, 15 mM NaCl, 0.1 mM EDTA, final 45 μM of each DNA strand). Then the solution was warmed up and cooled slowly (from 95 to 25° C. in 1 h, step gradient −5° C./˜4 min).

The template for the transcription of RNA having SP6 sequence (RNA7-9) was prepared according to the known procedure [8]

### Preparation of Template DNA for In Vitro Transcription of RNA Having V5x3, G276, Gluc, Egfp and Fluc Sequences

Template DNA for transcription of the RNA having 3xV5 sequence was prepared by digesting the 3xV5_ pUC57 plasmid with Aarl (Thermo) restriction enzyme. The plasmid 3xV5_pUC57 was made by GeniScript by cloning the gene of the following sequence into the pUC57 vector using the EcoRV strategy:

CAC GCT GTG TAA TAC GAC TCA CTA TAG GGG TAC
GCC ACC ATG GAA GGT AAG CCT ATC CCT AAC CCT
CTC CTC GGT CTC GAT TCT ACG GGC AGC AGC GGC
GGC AAA CCG ATT CCG AAC CCG CTG CTG GGC CTG
GAT AGC ACC GGT AGC AGC GGC GGT AAG CCT ATC
CCT AAC CCT CTC CTC GGT CTC GAT TCT ACG GTT
TAA ACA AAA AAA AAA AAA AAA AAA AAA AAA AAA
AAA AAA AAA AAA AAA AAA AAA AAA AAA AAA AAG
CAG GTG TCT AGA

Template DNA for RNA transcription of the G276 sequence was prepared by digesting the hRLuc-pRNA2(A)128 plasmid with Adel restriction enzyme (Thermo). The hRLuc-pRNA2(A)128 plasmid was made according to a known procedure [26].

Template DNA for RNA transcription of the gluc sequence was prepared by digesting the pJET1.2_T7_Gluc_3′utr-beta-globin_A128 plasmid with Aarl restriction enzyme (Thermo). The pJET1.2_T7_Gluc_3′utr-beta-globin_A128 was made according to the known procedure [11].

Template DNA for RNA transcription of the egfp sequence was prepared by digesting the pJET1.2_T7_Egfp_3′utr-beta-globin_A128 plasmid with Aarl restriction enzyme (Thermo). pJET1.2_T7_Egfp_3′utr-beta-globin_A128 was made in the same way as the pJET1.2_T7_Gluc_3′utr-beta-globin_A128 plasmid, by cloning the eGFP gene into the pJET1.2 vector [11].

Template DNA for RNA transcription of the fluc sequence was prepared by digesting the pJET1.2_T7_Fluc3′utr-beta-globin_A128 plasmid with Aarl restriction enzyme (Thermo). pJET1.2_T7_Fluc_3′utr-beta-globin_A128 was made according to the known procedure [8].

### In Vitro Transcription and Isolation of the RNA Encoding the A35 Sequence (RNA1, RNA3, RNA6)

RNA3: Reagents were added to the template DNA solution (45 μM, 11 μL) to obtain a reaction mixture (250 μL) with the following composition: Transcription buffer (x1, Thermo), GTP (5.0 mM), UTP (5.0 mM), CTP (5.0 mM), ATP (3.0 mM), N₃-AG (6.0 mM), MgCl₂ (20 mM), Ribolock (1 U/μL, Thermo), T7 RNAP (0.125 mg/mL). After incubation for 2 h at 37° C., DNase I (2 μL, 30 min, Thermo) was added and the incubation continued for another 30 min. Then an EDTA solution (250 μL, 30 mM) was added and deproteinization (washing of the reaction mixture with PhOH/CHCl₃ system, followed by CHCl₃, 1:1 v/v) and precipitation (50 μL of 3M NaOAc, pH 5.2/1.1 mL 99% EtOH, −20° C., ON) were performed. After centrifugation (>10 g, 20 min @4° C.), washing (80% EtOH) and drying in vacuo, the RNA pellet was dissolved in water (200 μL). Separation of the products by HPLC was performed: Phenomenex Clarity 3 μm Oligo RP C18 column, 50×4.6 mm. Buffers A: 50 mM TEAA, B: MeCN. Program: 5% B for 5 min, 5-10% B in 15 min, 10-50% B in 1 min, 50% for 4 min, flow 1.0 mL/min, @50° C. (RT˜16 min). The collected fractions were lyophilized, dissolved in water and separated on a PAA gel (15% PAA, 8M urea, 1×TBE) in order to analyze their composition. Fractions containing the desired RNA product were combined, lyophilized and dissolved in water. The RNA in the solution was precipitated in ethanol (as the sodium salt as before) and redissolved in water (160 μL). The concentration of the RNA product was measured with a nanodrop (A₂₆₀=2.48, 1.0 mm optical path). The product RNA3 (159 μg, 9.1 nmol, 3.98 mOD₂₆₀, E₂₆₀=437 mM⁻¹cm⁻¹) was obtained as a mixture of n-mers (˜35-36 nt).

RNA1: RNA1 transcription and isolation were performed as described for RNA3, except that the reaction mixture contained the following concentrations of selected reagents: ATP (5.0 mM), N₃-AG (0 mM).

RNA6: RNA6 transcription and isolation were performed as described for RNA3, except that the reaction mixture contained the following concentrations of selected reagents: EDA-AG (46.0 mM), N₃-AG (0 mM).

### In Vitro Transcription and Isolation of the RNA Encoding the SP6 Sequence (RNA7, RNA9)

RNA7: RNA7 transcription and isolation were performed according to a known procedure [8]

RNA9: RNA9 transcription and isolation were performed as described for RNA7, except that the reaction mixture contained the following concentrations of selected reagents: EDA-m⁷Gp₃G (1.0 mM), N₃-m⁷Gp₃G (0 mM).

### In Vitro Transcription and Isolation of RNA Encoding the V5x3, G276, Gluc, Egfp and Fluc Sequences (RNA10, RNA15, RNA16, RNA20, RNA24)

RNA20: Reagents were added to the template DNA solution (13 μg, 20 μL) to form a reaction mixture (130 μL) with the following composition: Transcription buffer (x1, Thermo), ATP (5.0 mM), UTP (5.0 mM), CTP (5.0 mM), GTP (1.0 mM), N₃-m⁷Gp₃G (6.0 mM), MgCl₂ (20 mM), Ribolock (1 U/μL, Thermo), T7 RNAP (0.125 mg/mL). After incubation for 135 min at 37° C., DNase I (2 μL, 30 min, Thermo) was added and incubations continued for another 30 min. Then EDTA solution (8 μL, 0.5 M) and water (420 μL) were added. Reaction products were purified with NucleoSpin® RNA (MACHEREY-NAGEL): 1 prep, loading in three portions, elution 2×60 μL. An RNA20 solution was obtained (152 μg/115 μL, 1.32 μg/μL, 2.31 μM). For further purification, portion of the obtained RNA was separated by HPLC chromatography: RNASept™ Prep C18 column, 50×7.8 mm, 2 μm, A—100 mM TEAOAc pH 7.0, B—200 mM TEAOAc pH 7.0/MeCN 1:1, 0.9 mL/min @55° C. Program: 18-30% B in 40 minutes The collected fractions were divided into ˜700 μL aliquots, NaOAc (70 μL, 3 M), glycogen (1 μL, 5 mg/mL) and iPrOH (800 μL) were added and incubated at −80° C. for 30 min. The pellets were centrifuged (30 min, 4° C., 15,000 g), supernatants were carefully removed, EtOH (80%, 0.5 mL) was added, the pellets were centrifuged again (10 min, 4° C., 14,000 g), dried under vacuum and dissolved in water (20 μL per sample). Product samples (60 ng) were separated on an agarose gel (1%, 1×TBE, 80 V 60 min). After combining the fractions containing the desired product, high-quality RNA20 was obtained (4.76 μg, 53%).

RNA10: Transcription on the appropriate DNA template (V5X3) and RNA10 isolation were performed as described for RNA20, except that the reaction mixture contained the following concentrations of selected reagents: GTP (5.0 mM), N₃-m⁷Gp₃G (0 mM). Different HPLC chromatography conditions were also used: SecurityGuard™ Cartridge Gemini®-NX C18 pre-column, 4×3.00 mm, +Phenomenex Clarity 3 μm Oligo RP C18 column, 150×4.6 mm, A—100 mM TEAOAc pH 7. RNA10: Transcription on the appropriate DNA template (V5X3) and RNA10 isolation were performed as described for RNA200, B—200 mM TEAOAc pH 7.0/MeCN 1:1, 1 mL/min @50° C. Program: 10-60% B in 60 min.

RNA12: transcription on the appropriate DNA template (G276) and RNA12 isolation were performed as described for RNA20, except that different HPLC chromatography conditions were used: SecurityGuard™ Cartridge Gemini®-NX C18 pre-column, 4×3.00 mm, +Phenomenex Clarity 3 μm Oligo RP C18 column, 150×4.6 mm, A—100 mM TEAOAc pH 7.0, B—200 mM TEAOAc pH 7.0/MeCN 1:1, 1 mL/min @50° C. Program: 10-60% B in 60 min.

RNA15: transcription on the appropriate DNA template (gluc) and RNA15 isolation were performed as described for RNA20, except that the reaction mixture contained the following concentrations of selected reagents: GTP (5.0 mM), N₃-m⁷Gp₃G (0 mM).

RNA16: transcription on the appropriate DNA template (gluc) and RNA16 isolation were performed as described for RNA20.

RNA24: transcription on an appropriate DNA template (fluc) and RNA24 isolation were performed as described for RNA20, except that the reaction mixture contained the following concentrations of selected reagents: GTP (1.0 mM), m₂^{3′-O,7}Gp₃G (6.0 mM) [27].

### Preparation and Isolation of RNA Labeled According to the Invention at the 3′ End of Cy3 (RNA2, RNA4, RNA11, RNA13, RNA17, RNA21, RNA25)

RNA21: Fresh NaIO₄ solution (2 μL, 10 mM) was added to the RNA20 solution (11.43 μg/12 μL) and incubated for 30 min at 25° C. Then KH₂PO₄ buffer (2 μL, 1M, pH 6.0), fresh NaBH₃CN solution (2 μL, 200 mM) and Cy3-EDA (2 μL, 10 mM, 50% DMSO) were added. After incubation for 120 min at 25° C., water (160 μL), NaOAc (20 μL, 3M, pH 5.9), glycogen (1 μL, 5 mg/mL) and EtOH (100%, 600 μL) were added and incubated at −80° C. for 30 minutes. The pellet was centrifuged (30 min, 4° C., 15,000 g), the supernatant was carefully removed, EtOH (80%, 800 μL) was added, the pellet was centrifuged again (10 min, 25° C., 14,000 g), dried under vacuum and dissolved in water (100 μL). RNA21 solution (101.1 ng/μL, 10.1 μg, 88%) was obtained. For further purification, a portion of the obtained RNA was separated by HPLC chromatography, as described for RNA20. After combining the fractions containing the desired product, high-quality RNA21 (3.30 μg, 37%) was obtained.

RNA2: The labeling reaction and RNA2 isolation were performed as described for RNA21, using RNA1 as substrate. HPLC chromatography conditions: Phenomenex Clarity 3 μm Oligo RP C18 column, 50×4.6 mm, A—50 mM TEAOAc pH 7.0, B MeCN, 1 mL/min @50° C. Program: 5-75% B in 10 min.

RNA4: The labeling reaction and RNA4 isolation were performed as described for RNA21, using RNA3 as substrate. HPLC chromatography conditions: as described for RNA2.

RNA11: The labeling reaction and RNA11 isolation were performed as described for RNA21, using RNA10 as substrate. HPLC conditions: as described for RNA10.

RNA17: The labeling reaction and RNA17 isolation were performed as described for RNA21, using RNA16 as substrate.

RNA25: The labeling reaction and RNA25 isolation were performed as described for RNA21, using RNA24 as substrate.

### Preparation and Isolation of RNA Labeled at the 5′ End of Cy5 (RNA18, RNA22)

RNA22: Buffer KH₂PO₄ (2 μL, 1M, pH 6.0) and DIBAC-sCy5 (2 μL, 20 mM, 50% DMSO, Lumiprobe) were added to the RNA20 solution (11.43 μg/16 μL). After incubation for 120 min at 25° C., water (160 μL), NaOAc (20 μL, 3M, pH 5.9), glycogen (1 μL, 5 mg/mL) and EtOH (100% 600 μL) were added and incubated at −80 ° C. for 30 minutes. The pellet was centrifuged (30 min, 4° C., 15,000 g), the supernatant was carefully removed, EtOH (80%, 800 μL) was added, the pellet was centrifuged again (10 min, 25° C., 14,000 g), dried under vacuum and dissolved in water (100 μL). RNA22 solution (94.3 ng/μL, 9.43 μg, 83%) was obtained. For further purification, a portion of the obtained RNA was separated by HPLC chromatography, as described for RNA20. After combining the fractions containing the desired product, high-quality RNA22 (4.78 μg, 53%) was obtained.

RNA18: The labeling reaction and RNA18 isolation were performed as described for RNA22, using RNA16 as substrate.

### Preparation and Isolation of RNA Labeled According to the Invention at the 3′ End of Cy3 and the 5′ End of Cy5 (RNA5, RNA8, RNA14, RNA19, RNA23)

RNA23: Fresh NaIO4 solution (2 μL, 10 mM) was added to the RNA20 solution (11.43 μg/10 μL) and incubated for 30 min at 25° C. Then KH₂PO₄ buffer (2 μL, 1M, pH 6.0), fresh NaBH₃CN solution (2 μL, 200 mM), DIBAC-sCy5 (2 μL, 20 mM, 50% DMSO, Lumiprobe) and Cy3-EDA (2 μL, 10 mM, 50% DMSO) were added. After incubation for 120 min at 25° C., water (160 μL), NaOAc (20 μL, 3M, pH 5.9), glycogen (1 μL, 5 mg/mL) and EtOH (100%, 600 μL) were added and incubated at −80° C. for 30 minutes. The pellet was centrifuged (30 min, 4° C., 15,000 g), the supernatant was carefully removed, EtOH (80%, 800 μL) was added, the pellet was centrifuged again (10 min, 25° C., 14,000 g), dried under vacuum and dissolved in water (100 μL). The RNA23 solution (94.6 ng/μL, 9.46 μg, 83%) was obtained. For further purification, a portion of the obtained RNA was separated by HPLC chromatography, as described for RNA20. After combining the fractions containing the desired product, high-quality RNA23 (2.66 μg, 37%) was obtained.

RNA5: The labeling reaction and RNA5 isolation were performed as described for RNA23, using RNA3 as substrate. HPLC chromatography conditions: Phenomenex Clarity 3 μm Oligo RP C18 column, 50×4.6 mm, A—50 mM TEAOAc pH 7.0, B MeCN, 1 mL/min @50° C. Program: 5-30% B in 20 min.

RNA8: The labeling reaction and RNA8 isolation were performed as described for RNA23, using RNA7 as substrate. HPLC conditions: as described for RNA5.

RNA14: The labeling reaction and RNA14 isolation were performed as described for RNA23, using RNA12 as substrate. HPLC conditions: as described for RNA10.

RNA19: The labeling reaction and RNA19 isolation were performed as described for RNA23, using RNA16 as substrate.

### Monitoring the Activity of RNase A, RNase T1 and RNAse R

The reaction buffer (4 mM Tris-HCl pH 7.5, 15 mM NaCl, 0.1 mM EDTA) was degassed under reduced pressure. A concentrated labeled RNA solution (RNA5) was then mixed with the buffer to obtain an RNA concentration suitable for fluorescence measurements (˜100 nM). RNA solution (50 μL) was warmed and slowly cooled down (from 95 to 25° C. in 1 h, step gradient −5° C./˜4 min) then incubated on ice in the dark. After dilution with degassed buffer (150 μL) or RiboLock RNase inhibitor buffer (Thermo, 10 μL+140 μL), the solution was placed in a quartz cuvette (1×1×350 mm) and the fluorescence spectrum was recorded (excitation 500 nm, range 510-850 nm, averaged over three spectra, 10 nm slit). The changes in the emission spectrum were measured at 5° C. After the system stabilized (5-15 min), the enzyme was added in the appropriate concentration:

RNase A (Thermo): 10 mg/mL stock solution, 1 μL of the million-fold diluted (˜10 ng/ml, H₂O) enzyme was added to the cuvette

RNase T1 (Thermo): 1000 U/μL stock solution, 1 μL of the 100-fold diluted (10 U/μL, H₂O) enzyme was added to the cuvette

RNase R (ABM): 10 U/μL stock solution, 1 μL of an enzyme was added to the cuvette The changes in the emission spectrum were then measured at 5° C. as the reaction progressed.

### Monitoring of Dcp1/2 Enzyme Activity

The reaction buffer (4 mM Tris-HCl pH 7.5, 15 mM NaCl, 0.1 mM EDTA) was degassed under reduced pressure. The concentrated RNA solution (RNA8) was then mixed with the buffer to obtain a probe solution (˜100 nM) for fluorescence measurements. The FRET probe solution (40 μL) was warmed and cooled slowly (from 95 to 25° C. in 1 h, step gradient −5° C./˜4 min) and then incubated on ice in the dark. After dilution with degassed buffer (150 μL), MgCl₂ (1 μL, 1M) was added, transferred to a quartz cuvette (1×1×350 mm) and the fluorescence spectrum was recorded (excitation 500 nm, range 510-850 nm, averaged over three spectra, slit 10 nm). The changes in the emission spectrum were measured at 5° C. After the system stabilized (5-15 min), Dcp1/2 complex with Schizosaccharomyces pombe (10 μL, 7 μM) was added. The changes in the emission spectrum were then measured at 5° C. as the reaction progressed.

### Monitoring of RNase H Activity

DNA solution (6.00 μL, 1 μM, 1.2 eq) was added to the FRET probe solution (RNAS, ˜100 nM, 50 μL) in buffer (412 μL; 4 mM Tris-HCl pH 7.5, 15 mM NaCl, 0.1 mM EDTA) or water (6.00 μL), warmed up and slowly cooled (from 95 to 25° C. in 1 h, step gradient −5° C./˜4 min). Then degassed water (160 μL) and Rnase H Buffer×10 (24 μL, 200 mM Tris-HCl pH 7.5, 500 mM NaCl, 100 mM MgCl₂, 10 mM DTT) were added and placed in a quartz cuvette (240 μL, 1×1×350 mm). The changes of the emission spectrum were measured at 35° C. (excitation 500 nm, range 510-850 nm, averaged over three spectra, slit 10 nm). After the system stabilized (2-5 min), RNase H (2.00 μL, 0.1 mg/mL) was added and the changes in the emission spectrum were measured at temperature as the reaction progressed.

### BIBLIOGRAPHY

[1] D. A. Baum, S. K. Silverman Angewandte Chemie International Edition 2007, 46, 3502
[2] L. Büttner, F. Javadi-Zarnaghi, C. Höbartner Journal of the American Chemical Society 2014, 136, 8131
[3] M. G. Maghami, C. P. M. Scheitl, C. Höbartner Journal of the American Chemical Society 2019, 141, 19546
[4] M. G. Maghami, S. Dey, A. Lenz, C. Höbartner Angewandte Chemie International Edition 2020, 59, 9335
[5] B. Samanta, D. P. Horning, G. F. Joyce Nucleic Acid Research 2018, 46, e103
[6] T. E. England, O. E. Uhlenbeck Nature 1978, 275, 560
[7] W. C. Lai, M. Kayedkhordeh, E. V. Cornell, E. Farah, S. Bellaousov, R. Rietmeijer, E. Salsi, D. H. Mathews, D. N. Ermolenko Nature Communications 2018, 9, 4328
[8] A. Mamot, P. J. Sikorski, M. Warminski, J. Kowalska, J. Jemielity Angewandte Chemie International Edition 2017, 56, 15628
[9] S. Croce, S. Serdjukow, T. Carell, T. Frischmuth ChemBioChem 2019, 21, 1641
[10] L. Anhäuser, S. Hüwel, T. Zobel, A. Rentmeister Nucleic Acid Research 2019, 47, e42
[11] P. S. Sikorski, M. Warminski, D. Kubacka, T. Ratajczak, D. Nowis, J. Kowalska, J. Jemielity Nucleic Acid Research 2020, 48, 1607
[12] D. Strzelecka, M. Smietanski, P. J. Sikorski, M. Warminski, J. Kowalska, J. Jemielity RNA 2020, 26, 1815
[13] M. Tomkuvienė, M. Mickutė, G. Vilkaitis, S. Klimašauskas Current Opinion in Biotechnology 2019, 55, 114
[14] L. Bellon, C. Workman, J. Scherrer, N. Usman, F. Wincott Journal of the American Chemical Society 1994, 118, 3771
[15] N. Rublack, S. Müller Beilstein Journal of Organic Chemistry 2014, 10, 1906
[16] G. Lentzen, B. Dobberstein, W. Wintermeyer FEBS Letters 1994, 348, 233
[17] E. Griffin, Z. Qin, W. J. Michels, A. M. Pyle Chemistry & Biology 1995, 2, 761
[18] S. Busch, L. A. Kirsebom, H. Notbohm, R. K. Hartmann Journal of Molecular Biology 2000, 299, 941
[19] O. Rossbach, L. H. Hung, S. Schreiner, I. Grishina, M. Heiner, J. Hui, A. Bindereif Molecular and Cellular Biology 2009, 29, 1442
[20] D. Li, M. H. F. Meyer, D. K. Willkomm, M. Keusgen, R. K. Hartmann Biochemie 2010, 92, 772
[21] M. Zhao, F. D Steffen, R. Börner, M. F. Schaffer, R. K. O. Sigel, E. Freisinger Nucleic Acids Research 2018, 46, e13
[22] U. Ragnarson Chemical Society Reviews 2001, 30, 205
[23] M. R. Johnson, W. R. Thelin PCT/US2016/029729 WO 2016/176423
[24] P. Wanat, S. Walczak, B. A. Wojtczak, M. Nowakowska, J. Kowalska, J. Jemielity Organic Letters 2015, 17, 3062
[25] M. Warminski, J. Kowalska, J. Jemielity Organic Letters 2017, 19, 3624
[26] M. Warminski, P. J. Sikorski, Z. Warminska, M. Lukaszewicz, A. Kropiwnicka, J. Zuberek, E. Darzynkiewicz, J. Kowalska, J. Jemielity Biocojnugate Chemistry 2017, 28 1978
[27] J. Jemielity, T. Fowler, J. Zuberek, J. Stepinski, M. Lewdorowicz, A. Niedzwiecka, R. Stolarski, E. Darzynkiewicz, R. E. Rhoads RNA 2003, 9, 1108

1. An RNA analog of formula 1: 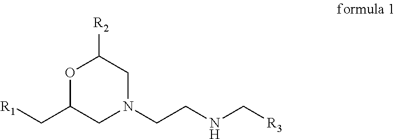
where:
R₁ is:
RNA chain of formula 2a: 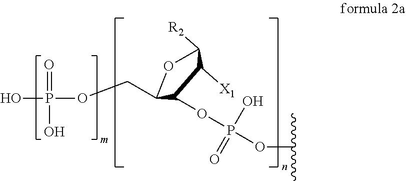
wherein:
n is a natural number in the range from 1 to 10,000,
m is a natural number in the range from 0 to 3,
X₁ is independently: OH or OCH₃,
or RNA chain of formula 2b: 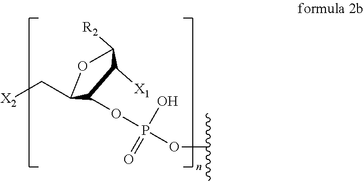
wherein:
n is a natural number in the range from 1 to 10,000,
X₁ is independently: OH or OCH₃,
X₂ is N₃ or 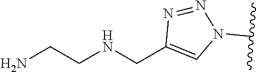 [SMILES: *n1cc(CNCCN)nn1]
group,
or an RNA chain of formula 2c: 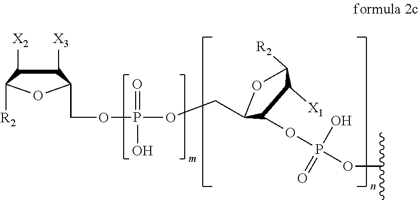
wherein:
n is a natural number in the range from 1 to 10,000,
m is a natural number in the range from 1 to 4,
X₁ is independently: OH or OCH₃
X₂ and X₃ are independently: OH, OCH₃, or 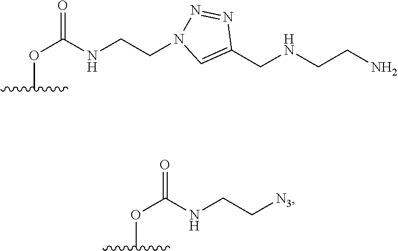 [SMILES: *OC(=O)NCCn1cc(CNCCN)nn1 ; *OC(=O)NCCN=[N+]=[N-]]
R₂ is a natural or modified purine or pyrimidine nitrogenous base, preferably selected from: 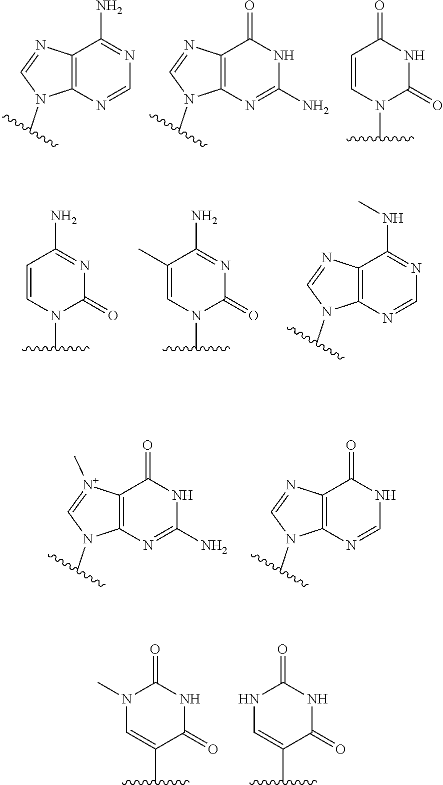
R₃ is a functional substituent such as:
a substituent containing a bioorthogonal group of formula 3a: 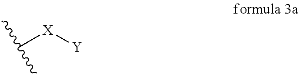
wherein
Y is NH₂, N₃ or 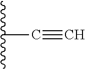 [SMILES: *C#C]
group
or a substituent having the structure of a fluorophore from the cyanine group of formula 3b: 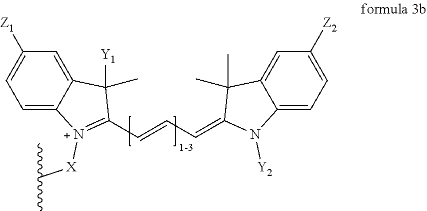
wherein:
Y₁ and Y₂ are independently: CH₃, (CH₂)₃SO₃H or (CH₂)₄SO₃H,
Z₁ and Z₂ are independently: SO₃H or H,
or a substituent having the structure of a fluorophore from the rhodamine or fluorescein group of formula 3c: 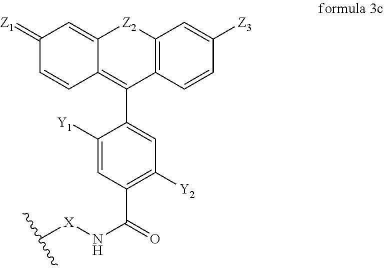
wherein:
Y₁ and Y₂ are independently: SO₃, OCH₃, OH, COOH or H,
Z₁ and Z₂ are independently: NH or O,
Z₃ is NH₂ or OH group,
or a substituent having the structure of a fluorophore from the rhodamine group of formula 3d: 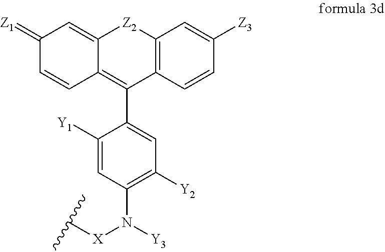
wherein:
Y₁ and Y₂ are independently: SO₃H, OCH₃, OH, COOH or H,
Y₃ is CH₂CH₃, CH₃ or H group,
Z₁ and Z₂ are independently: NH or O,
Z₃ is NH₂ or OH group,
or a substituent having an affinity tag structure of formula 3e: 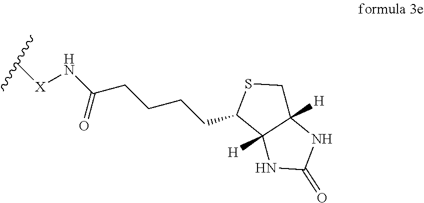
or a substituent having a nucleic acid structure of formula 3f: 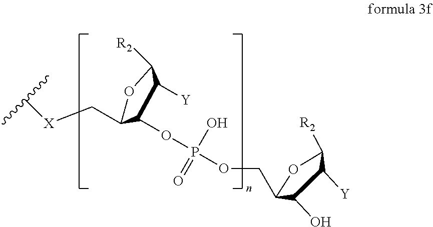
wherein:
Y is independently: OCH₃, OH or H,
n is a natural number in the range from 1 to 30,
R₂ is nitrogenous base as above,
or a substituent having a nucleic acid structure of formula 3g: 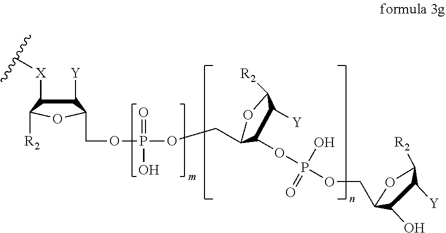
wherein:
Y is independently OCH₃, OH or H group,
m is a natural number in the range from 1 to 4,
n is a natural number in the range from 1 to 30,
R₂ is nitrogenous base as above,
or a substituent having a nucleic acid structure of formula 3h: 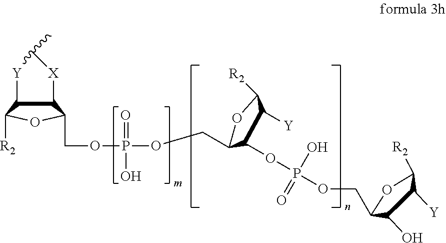
wherein:
Y is independently OCH₃, OH or H group,
m is a natural number in the range from 1 to 4,
n is a natural number in the range from 1 to 30,
R₂ is nitrogenous base as above,
wherein in the above formulas (3a to 3h) X is a linker of formula being any group or a serial combination of many of the following groups: 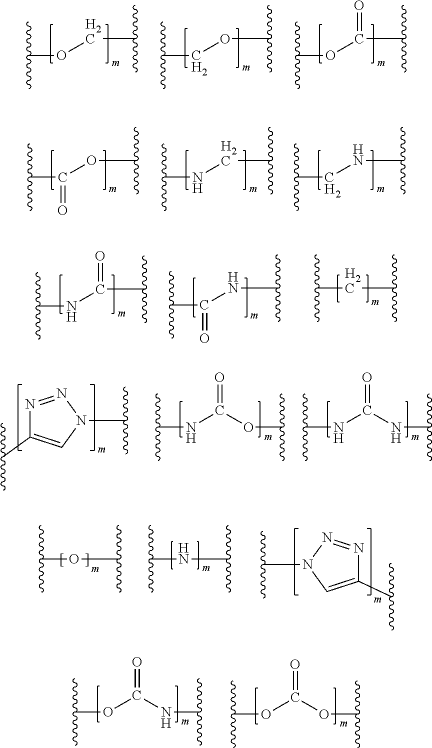
wherein m is a natural number ranging from 1 to 10.

2. A method for the preparation of an RNA analog of formula 1, as defined in claim 1, characterized in that the solution of RNA of formula 4: 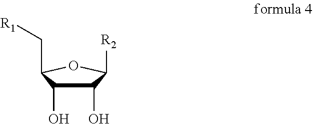
is subjected to successive:
(i) incubation with metaperiodic acid (HIO₄), with its salt, a solution of metaperiodic acid (HIO₄) or its salt, to provide a compound of formula 5: 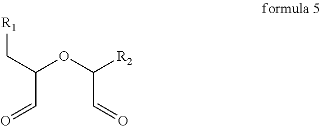
(ii) incubation in a reducing medium with an ethylenediamine analog of formula 6: 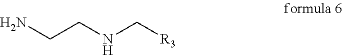
to give an RNA analog of formula 1: 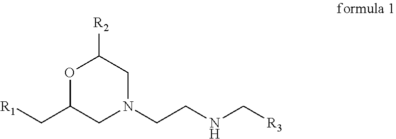
wherein the meaning of the groups R₁, R₂ and R₃ in the above formulas is defined in claim 1.

3. The method according to claim 2, characterized in that steps (i) and (ii) are carried out in one reactor.

4. The method according to claim 2, characterized in that the RNA is:
compound of formula 4a: 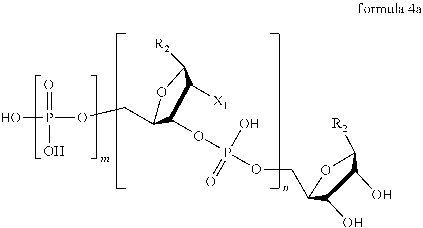
wherein:
n is a natural number in the range from 1 to 10,000,
m is a natural number in the range from 0 to 3,
X1 is independently: OCH3 or OH,
or compound of formula 4b. 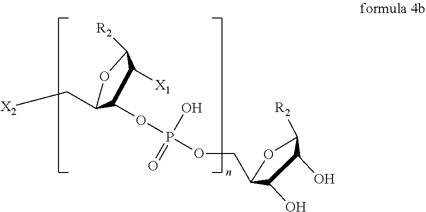
wherein:
n is a natural number in the range from 1 to 10,000,
X1 is independently: OH or CH3 group,
X₂ is N₃ or 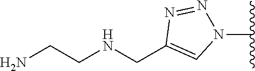 [SMILES: *n1cc(CNCCN)nn1]
group,
or a compound of formula 4c: 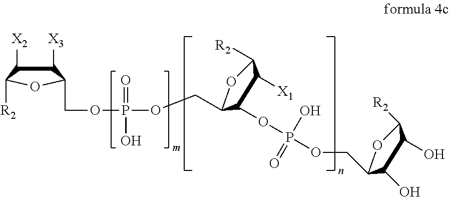
wherein:
n is a natural number in the range from 1 to 10,000,
m is a natural number in the range from 1 to 4,
X₁ is independently: OH or OCH₃
X₂ and X₃ are independently: OH, OCH₃,

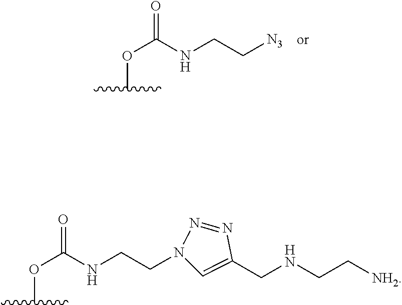 [SMILES: *OC(=O)NCCN=[N+]=[N-] ; *OC(=O)NCCn1cc(CNCCN)nn1]

5. The method according to claim 2, characterized in that step (i) is carried out in the presence of NaIO₄, preferably at a concentration of 1.0 to 1.5 mM, at a temperature below 40° C., and at the RNA concentration of 1 to 100 μM.

6. The method according to claim 2,
characterized in that step (ii) is carried out in the presence of a KH₂PO₄ buffer, preferably at pH 5.5-7.5, a NaBH₃CN reducing agent at a concentration not exceeding 100 mM, preferably at a concentration of 20 mM, and an ethylenediamine analog at a concentration of 1-10 mM.

7. The method according to claim 2, characterized in that the obtained RNA analog is isolated from the reaction mixture by a known method of RNA isolation, preferably by alcohol precipitation of the RNA salt or by means of high-performance liquid chromatography (HPLC).

8. Ethylenediamine analog of formula 7: 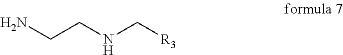
wherein R is:
a substituent having the structure of a fluorophore from the cyanine group of the formula 7a: 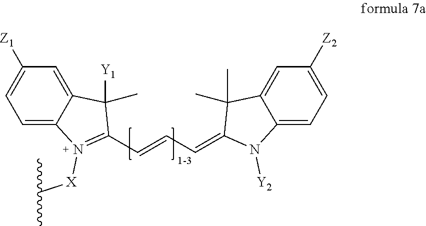
wherein:
Y₁ and Y₂ are independently: CH₃, (CH₂)₃SO₃H or (CH₂)₄SO₃H,
Z₁ and Z₂ are independently: SO₃H or H,
or a substituent having the structure of a fluorophore from the rhodamine or fluorescein group of formula 7b: 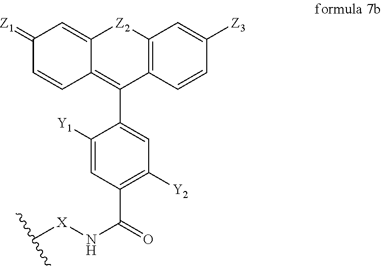
wherein:
Y₁ and Y₂ are independently: SO₃H, OCH₃, OH, COOH or H,
Z₁ and Z₂ are independently: NH or O,
Z₃ is NH₂ or OH group,
or a substituent having the structure of a fluorophore from the rhodamine group of formula 7c: 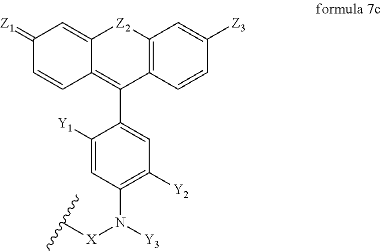
wherein:
Y₁ and Y₂ are independently: SO₃H, OCH₃, OH, COOH or H,
Y₃ is CH₂CH₃, CH₃ or H group,
Z₁ and Z₂ are independently: NH or O,
Z₃ is NH₂ or OH group,
or a substituent having an affinity tag structure of formula 7d: 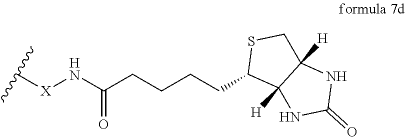
or a substituent having a nucleic acid structure of formula 7e: 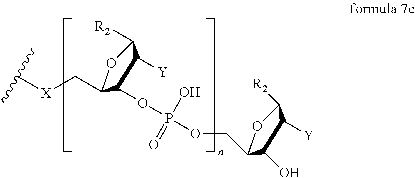
wherein:
Y is independently OCH₃, OH or H group,
n is a natural number in the range from 1 to 30,
R₂ is nitrogenous base as above,
or a substituent having a nucleic acid structure of formula 7f: 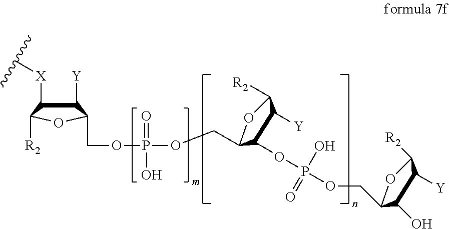
wherein:
Y is independently OCH₃, OH or H group,
m is a natural number in the range from 1 to 4,
n is a natural number in the range from 1 to 30,
R₂ is nitrogenous base as above,
or a substituent having a nucleic acid structure of formula 7g: 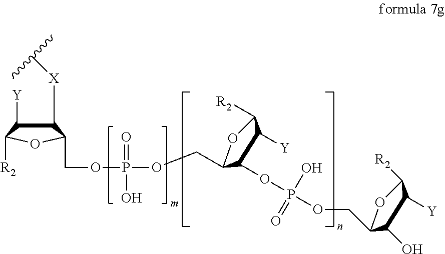
wherein:
Y is independently OCH₃, OH or H group,
m is a natural number in the range from 1 to 4,
n is a natural number in the range from 1 to 30,
R2 is nitrogenous base as above,
wherein in the above formulas (7a to 7g) X is a linker of formula being any group or a serial combination of many of the following groups: 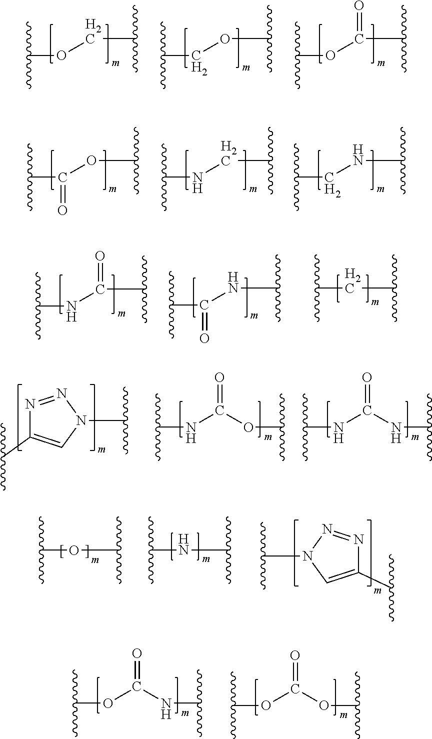
wherein m is a natural number ranging from 1 to 10.

9. The ethylenediamine analog according to claim 8, characterized in that it is selected from the compounds of the formulas:

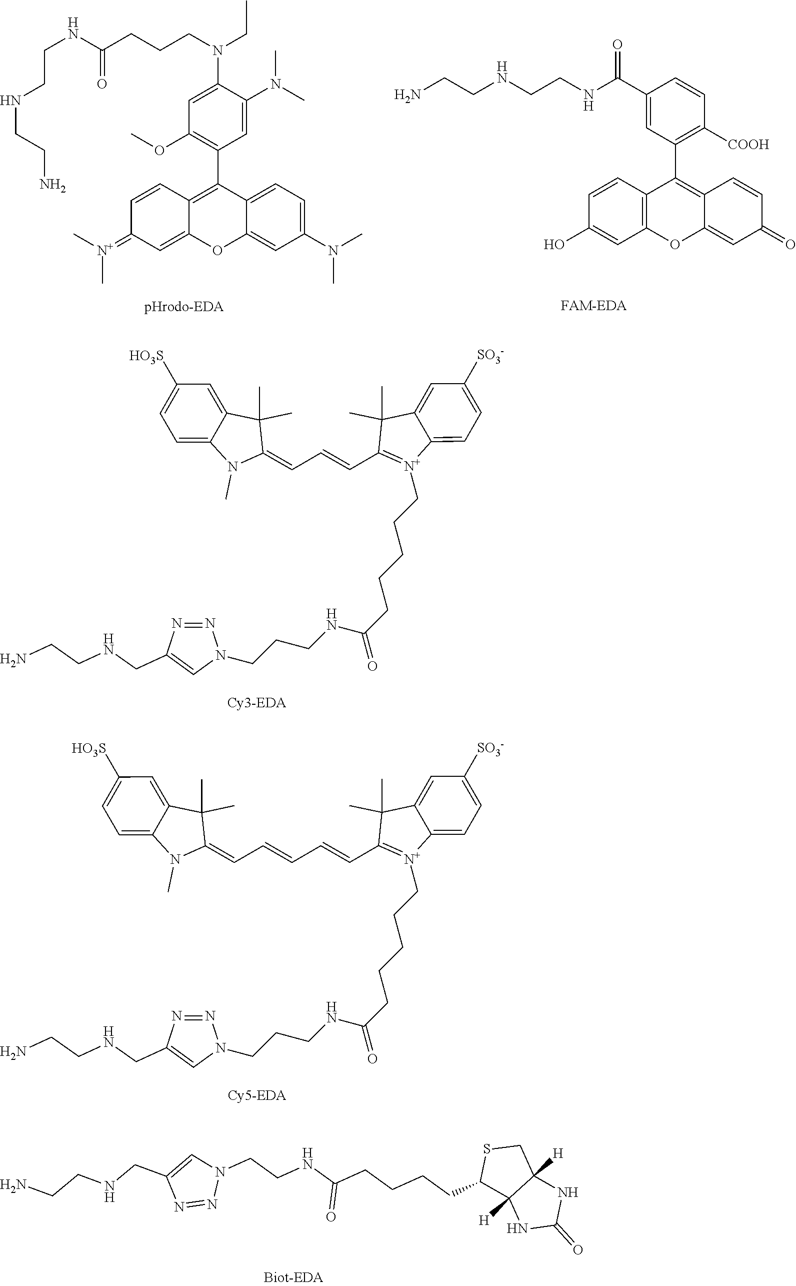

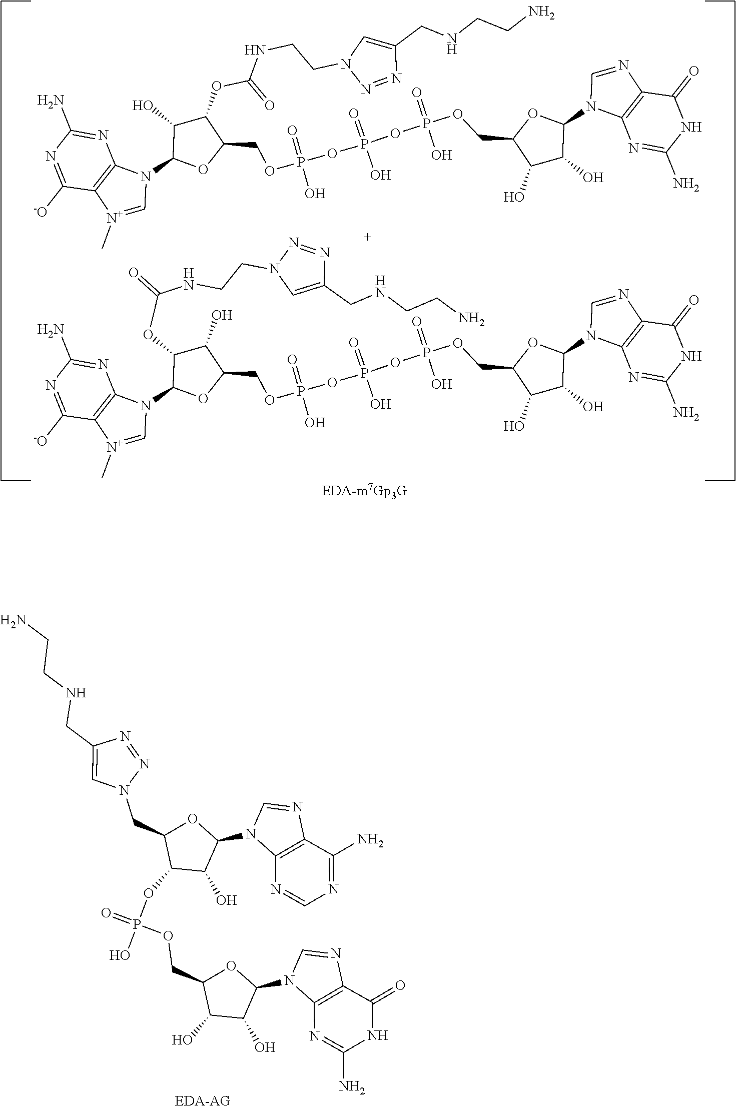

10. The method according to claim 3, characterized in that the RNA is: compound of formula 4a: 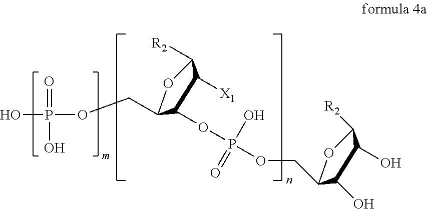
wherein:
n is a natural number in the range from 1 to 10,000,
m is a natural number in the range from 0 to 3,
X1 is independently: OCH3 or OH,
or compound of formula 4b: 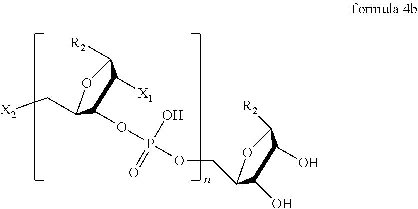
wherein:
n is a natural number in the range from 1 to 10,000,
X1 is independently: OH or CH3 group,
X₂ is N₃ or 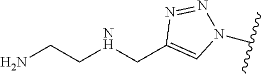 [SMILES: *n1cc(CNCCN)nn1]
group,
or a compound of formula 4c: 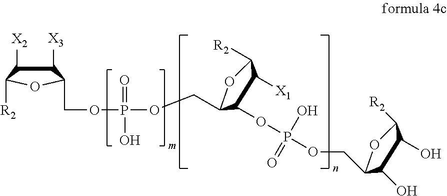
wherein:
n is a natural number in the range from 1 to 10,000,
m is a natural number in the range from 1 to 4,
X₁ is independently: OH or OCH₃
X₂ and X₃ are independently: OH, OCH₃,

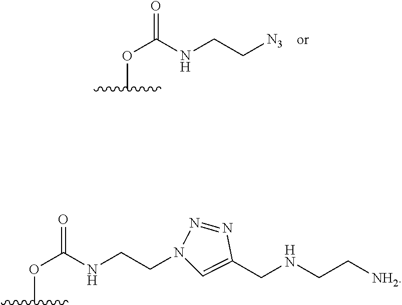 [SMILES: *OC(=O)NCCN=[N+]=[N-] ; *OC(=O)NCCn1cc(CNCCN)nn1]

11. The method according to claim 3, characterized in that step (i) is carried out in the presence of NaIO₄, preferably at a concentration of 1.0 to 1.5 mM, at a temperature below 40° C., and at the RNA concentration of 1 to 100 μM.

12. The method according to claim 4, characterized in that step (i) is carried out in the presence of NaIO₄, preferably at a concentration of 1.0 to 1.5 mM, at a temperature below 40° C., and at the RNA concentration of 1 to 100 μM.

13. The method according to claim 3, characterized in that step (ii) is carried out in the presence of a KH₂PO₄ buffer, preferably at pH 5.5-7.5, a NaBH₃CN reducing agent at a concentration not exceeding 100 mM, preferably at a concentration of 20 mM, and an ethylenediamine analog at a concentration of 1-10 mM.

14. The method according to claim 4, characterized in that step (ii) is carried out in the presence of a KH₂PO₄ buffer, preferably at pH 5.5-7.5, a NaBH₃CN reducing agent at a concentration not exceeding 100 mM, preferably at a concentration of 20 mM, and an ethylenediamine analog at a concentration of 1-10 mM.

15. The method according to claim 5, characterized in that step (ii) is carried out in the presence of a KH₂PO₄ buffer, preferably at pH 5.5-7.5, a NaBH₃CN reducing agent at a concentration not exceeding 100 mM, preferably at a concentration of 20 mM, and an ethylenediamine analog at a concentration of 1-10 mM.

16. The method according to claim 3, characterized in that the obtained RNA analog is isolated from the reaction mixture by a known method of RNA isolation, preferably by alcohol precipitation of the RNA salt or by means of high-performance liquid chromatography (HPLC).

17. The method according to claim 4, characterized in that the obtained RNA analog is isolated from the reaction mixture by a known method of RNA isolation, preferably by alcohol precipitation of the RNA salt or by means of high-performance liquid chromatography (HPLC).

18. The method according to claim 5, characterized in that the obtained RNA analog is isolated from the reaction mixture by a known method of RNA isolation, preferably by alcohol precipitation of the RNA salt or by means of high-performance liquid chromatography (HPLC).

19. The method according to claim 6, characterized in that the obtained RNA analog is isolated from the reaction mixture by a known method of RNA isolation, preferably by alcohol precipitation of the RNA salt or by means of high-performance liquid chromatography (HPLC).


SEQUENCE LISTING (30 sequences)

SEQ ID NO: 1 - DNA, 55 bp, artificial
  A35 oligo 1
cagtaatacgactcactattagggaagcgggcatgcggccagccatagccgatca

SEQ ID NO: 2 - DNA, 55 bp, artificial
  A35 oligo 2
tgatcggctatggctggccgcatgcccgcttccctaatagtgagtcgtattactg

SEQ ID NO: 3 - DNA, 276 bp, artificial
  cDNA 3xV5
cacgctgtgtaatacgactcactataggggtacgccaccatggaaggtaagcctatccctaaccctctcctcggtctcgattctacgggcagcagcggcggcaaaccgattccgaacccgctgctgggcctggatagcaccggtagcagcggcggtaagcctatccctaaccctctcctcggtctcgattctacggtttaaacaaaaaaaaaaaaaaaaaaaaaaaaaaaaaaaaaaaaaaaaaaaaaaaaaaaaaaaaaaaagcaggtgtctaga

SEQ ID NO: 4 - DNA, 35 bp, artificial
  A35
agggaagcgggcatgcggccagccatagccgatca

SEQ ID NO: 5 - DNA, 35 bp, artificial
  SP6
gaagaagcgggcatgcggccagccatagccgatca

SEQ ID NO: 6 - RNA, 237 nt, artificial
  V5x3
gggguacgccaccauggaagguaagccuaucccuaacccucuccucggucucgauucuacgggcagcagcggcggcaaaccgauuccgaacccgcugcugggccuggauagcaccgguagcagcggcgguaagccuaucccuaacccucuccucggucucgauucuacgguuuaaacaaaaaaaaaaaaaaaaaaaaaaaaaaaaaaaaaaaaaaaaaaaaaaaaaaaaaaaaaaaa

SEQ ID NO: 7 - RNA, 276 nt, artificial
  G276
gggagagauaucacgcguucuagagcuagcgcuaccggacucagaucucgagcucaagcuuggcauuccgguacuguugguaaagccaccauggcuuccaagguguacgaccccgagcaacgcaaacgcaugaucacugggccucaguggugggcucgcugcaagcaaaugaacgugcuggacuccuucaucaacuacuaugauuccgagaagcacgccgagaacgccgugauuuuucugcaugguaacgcugccuccagcuaccuguggaggcac

SEQ ID NO: 8 - RNA, 993 nt, artificial
  gluc
ggggguacgccaccaugggagucaaaguucuguuugcccugaucugcaucgcuguggccgaggccaagcccaccgagaacaacgaagacuucaacaucguggccguggccagcaacuucgcgaccacggaucucgaugcugaccgcgggaaguugcccggcaagaagcugccgcuggaggugcucaaagaguuggaagccaaugcccggaaagcuggcugcaccaggggcugucugaucugccugucccacaucaagugcacgcccaagaugaagaaguucaucccaggacgcugccacaccuacgaaggcgacaaagaguccgcacagggcggcauaggcgaggcgaucgucgacauuccugagauuccuggguucaaggacuuggagcccuuggagcaguucaucgcacaggucgaucuguguguggacugcacaacuggcugccucaaagggcuugccaacgugcaguguucugaccugcucaagaaguggcugccgcaacgcugugcgaccuuugccagcaagauccagggccagguggacaagaucaagggggccgguggugacuaaggauccuagggccgcagcucgcuuucuugcuguccaauuucuauuaaagguuccuuuguucccuaaguccaacuacuaaacugggggauauuaugaagggccuugagcauuuggauucugccuaauaaaaaacauuuauuuucauugcguuuagcucgcuuucuugcuguccaauuucuauuaaagguuccuuuguucccuaaguccaacuacuaaacugggggauauuaugaagggccuugagcauuuggauucugccuaauaaaaaacauuuauuuucauugcauuuaaauguuuaaacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - RNA, 1155 nt, artificial
  egfp
ggggguacgccaccauggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguaaggauccuagggccgcagcucgcuuucuugcuguccaauuucuauuaaagguuccuuuguucccuaaguccaacuacuaaacugggggauauuaugaagggccuugagcauuuggauucugccuaauaaaaaacauuuauuuucauugcguuuagcucgcuuucuugcuguccaauuucuauuaaagguuccuuuguucccuaaguccaacuacuaaacugggggauauuaugaagggccuugagcauuuggauucugccuaauaaaaaacauuuauuuucauugcauuuaaauguuuaaacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - DNA, 2098 bp, artificial
  fluc
gggagtactgttggtaaagccaccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - DNA, 50 bp, artificial
  A0
tggctggccgcatgcccgcttcccttgatcggctatggctggccgcatgc

SEQ ID NO: 12 - DNA, 51 bp, artificial
  A1
tggctggccgcatgcccgcttccctatgatcggctatggctggccgcatgc

SEQ ID NO: 13 - DNA, 53 bp, artificial
  A3
tggctggccgcatgcccgcttccctaaatgatcggctatggctggccgcatgc

SEQ ID NO: 14 - DNA, 55 bp, artificial
  A5
tggctggccgcatgcccgcttccctaaaaatgatcggctatggctggccgcatgc

SEQ ID NO: 15 - DNA, 50 bp, artificial
  B1
tggctggccgcatgcccgcttcccaagatcggctatggctggccgcatgc

SEQ ID NO: 16 - DNA, 50 bp, artificial
  B3
tggctggccgcatgcccgcttcaaaaattcggctatggctggccgcatgc

SEQ ID NO: 17 - DNA, 50 bp, artificial
  B5
tggctggccgcatgcccgctaaaaaaataaggctatggctggccgcatgc

SEQ ID NO: 18 - DNA, 45 bp, artificial
  C1
tggctggccgcatgcccgcttcatcggctatggctggccgcatgc

SEQ ID NO: 19 - DNA, 47 bp, artificial
  C3
tggctggccgcatgcccgcttcaaatcggctatggctggccgcatgc

SEQ ID NO: 20 - DNA, 49 bp, artificial
  C5
tggctggccgcatgcccgcttcaaaaatcggctatggctggccgcatgc

SEQ ID NO: 21 - DNA, 52 bp, artificial
  A2
tggctggccgcatgcccgcttccctaatgatcggctatggctggccgcatgc

SEQ ID NO: 22 - DNA, 54 bp, artificial
  A4
tggctggccgcatgcccgcttccctaaaatgatcggctatggctggccgcatgc

SEQ ID NO: 23 - DNA, 50 bp, artificial
  B0a
tggctggccgcatgcccgcttccctagatcggctatggctggccgcatgc

SEQ ID NO: 24 - DNA, 50 bp, artificial
  B0b
tggctggccgcatgcccgcttcccatgatcggctatggctggccgcatgc

SEQ ID NO: 25 - DNA, 50 bp, artificial
  B1a
tggctggccgcatgcccgcttccctaaatcggctatggctggccgcatgc

SEQ ID NO: 26 - DNA, 50 bp, artificial
  B1b
tggctggccgcatgcccgcttccaatgatcggctatggctggccgcatgc

SEQ ID NO: 27 - DNA, 50 bp, artificial
  B2
tggctggccgcatgcccgcttccaaaaatcggctatggctggccgcatgc

SEQ ID NO: 28 - DNA, 51 bp, artificial
  D11
tggctggccgcatgcccgcttcccaaagatcggctatggctggccgcatgc

SEQ ID NO: 29 - DNA, 52 bp, artificial
  D22
tggctggccgcatgcccgcttccaaaaaaatcggctatggctggccgcatgc

SEQ ID NO: 30 - DNA, 53 bp, artificial
  D33
tggctggccgcatgcccgcttcaaaaaaaattcggctatggctggccgcatgc